(19) United States
(12) Patent Application Publication
(10) Pub. No.: US 2025/0127868 A1
(43) Pub. Date: Apr. 24, 2025

(54) HYPERACTIVATING LIPID NANOPARTICLES

(71) Applicant: Corner Therapeutics, Inc., Watertown, MA (US)

(72) Inventors: Emily A. GOSSELIN, West Roxbury, MA (US); Andrew N. CORNFORTH, Carlisle, MA (US); Jonathan CHOW, Newton, MA (US); Dania ZHIVAKI, West Roxbury, MA (US); Kelsey K. FINN, Cambridge, MA (US)

(73) Assignee: Corner Therapeutics, Inc., Watertown, MA (US)

(21) Appl. No.: 18/836,260

(22) PCT Filed: Feb. 6, 2023
(86) PCT No.: PCT/US2023/062064

§ 371 Date: Aug. 6, 2024

Related U.S. Application Data
(60) Provisional application No. 63/307,569, filed on Feb. 7, 2022.
(60) Provisional application No. 63/417,282, filed on Oct. 18, 2022.

(51) Int. Cl. A61K 39/00; A61K 9/51; A61K 39/39; A61K 40/19; A61P 37/04

(52) U.S. Cl. CPC A61K 39/0011; A61K 9/5123; A61K 39/39; A61K 40/19; A61P 37/04; A61K 2039/5252; A61K 2039/55505; A61K 2039/55566; A61K 2039/55577

(57) ABSTRACT

The present disclosure relates to lipid nanoparticles comprising a lysophosphatidylcholine (LPC) compound and at least one further lipid, and uses thereof in hyperactivating mammalian dendritic cells, such as human dendritic cells. The present disclosure also relates to compositions comprising lipid nanoparticles comprising a LPC and at least on further lipid, in which the compositions comprise one or more of a pathogen recognition receptor agonist, an antigen, and mammalian dendritic cells, as well as methods for production and use of the compositions.

### CROSS-REFERENCE TO RELATED APPLICATIONS

This application claims priority to and benefit of U.S. Provisional Patent Application No. 63/417,282, filed Oct. 18, 2022, and U.S. Provisional Patent Application No. 63/307,569, filed Feb. 7, 2022, each of which is incorporated herein by reference in its entirety.

### FIELD

The present disclosure relates to lipid nanoparticles comprising a lysophosphatidylcholine (LPC) compound and at least one further lipid, and uses thereof in hyperactivating mammalian dendritic cells, such as human dendritic cells. The present disclosure also relates to compositions comprising lipid nanoparticles comprising a LPC and at least one further lipid, in which the compositions comprise one or more of a pathogen recognition receptor agonist, an antigen, and mammalian cells, as well as methods for production and use of the compositions.

### BACKGROUND

Lipid nanoparticles (LNPs) have become important vaccine delivery tools, especially in the context of mRNA vaccines. While LNP-based mRNA vaccines, as well as protein subunit vaccines, are effective at inducing antigen-specific antibody responses, they often exhibit limited antigen-specific T cell responses.

Dendritic cells (DCs) provide T cells with several signals that are important for the establishment of appropriate T cell responses. The type and magnitude of the signals are dependent upon the activation states of the DCs (Zhivaki and Kagan, Nature Reviews Immunology, 22:322-339, 2022). Naive DCs are quiescent cells having the ability to take up antigens. Active DCs not only have the ability to take up antigens, but also have an enhanced ability to present peptide fragments of antigens on major histocompatibility complex molecules. Additionally, active DCs have increased expression of co-stimulatory molecules for stimulation of T cells. Hyperactive DCs share activities with their active DC counterparts, but also gain the abilities to hypermigrate to lymph nodes and to secrete IL-1β. Like hyperactive DCs, pyroptotic DCs secrete high levels of IL-1β. However, pyroptotic DCs are dead cells which quickly lose their T cell stimulatory capacity. When DCs are matured using the pathogen-associated molecular pattern (PAMP)-containing molecule, lipopolysaccharide (LPS) and the damage-associated molecular pattern (DAMP)-containing molecule such as PGPC (1-palmitoyl-2-glutaryl-sn-glycero-3-phosphocholine) they secrete IL-1β without pyroptosing, characterizing these cells as hyperactive (Zanoni et al., Science, 352 (6290): 1232-1236, 2016).

Accordingly, LNP formulations having the ability to hyperactivate dendritic cells are desirable for inclusion in vaccines. In particular, LNPs having the ability to induce IL-1β secretion and enhance the generation of long-lived T cell responses are needed in the art.

### BRIEF SUMMARY

The present disclosure relates to lipid nanoparticles comprising a lysophosphatidylcholine (LPC) compound and at least one further lipid, and uses thereof in hyperactivating mammalian dendritic cells. The present disclosure also relates to compositions comprising lipid nanoparticles comprising a LPC and at least one further lipid, wherein the compositions further comprise one or more of a pathogen recognition receptor agonist, an antigen, and mammalian dendritic cells, as well as methods for production and use of the compositions.

In particular, the present disclosure provides a composition comprising an isolated lysophosphatidylcholine (LPC) with a single acyl chain, at least one further lipid, and a TLR7/8 agonist, wherein the acyl chain is a C13-C22 acyl chain or a C13-C24 acyl chain, and the LPC and the at least one further lipid are part of a lipid nanoparticle (LNP). In some embodiments, the at least one further lipid is selected from the group consisting of an ionizable lipid, a cationic lipid, a further phospholipid, a pegylated lipid, a structural lipid, and mixtures thereof. In some embodiments, the acyl chain is a C18-C22 acyl chain, a C21-C24 acyl chain, or a C22 acyl chain. In some embodiments, the composition further comprises an antigen and/or dendritic cells.

In some aspects, the present disclosure provides a composition comprising an isolated lysophosphatidylcholine (LPC) with a single acyl chain, at least one further lipid, and an antigen, wherein the acyl chain is a C21-C24 acyl chain, and the LPC and the at least one further lipid are part of a lipid nanoparticle (LNP). In some embodiments, the at least one further lipid is selected from the group consisting of an ionizable lipid, a cationic lipid, a further phospholipid, a pegylated lipid, a structural lipid, and mixtures thereof. In some embodiments, the composition further comprises dendritic cells and/or a TLR7/8 agonist.

In some aspects, the present disclosure provides a composition comprising an isolated lysophosphatidylcholine (LPC) with a single acyl chain, at least one further lipid, and dendritic cells, wherein the acyl chain is a C21-C24 acyl chain and the LPC and the at least one further lipid are part of a lipid nanoparticle (LNP). In some embodiments, the at least one further lipid is selected from the group consisting of an ionizable lipid, a cationic lipid, a further phospholipid, a pegylated lipid, a structural lipid, and mixtures thereof. In some embodiments, the composition further comprises a TLR7/8 agonist and/or an antigen.

In some embodiments of the preceding aspects, the antigen is present in a biological sample obtained from an individual. In some embodiments, the biological sample comprises biopsy tissue. In some embodiments, the biological sample comprises cells. In other embodiments, the biological sample does not comprise cells. In some embodiments, the biological sample comprises pus from an abscess. In some embodiments, the antigen comprises a proteinaceous antigen. In some embodiments, the antigen comprises a tumor antigen. In some embodiments, the tumor antigen comprises a synthetic or recombinant neoantigen. In some embodiments, the tumor antigen comprises a tumor cell lysate. In some embodiments, the antigen comprises a microbial antigen and the microbial antigen comprises one or more of a viral antigen, a bacterial antigen, a protozoan antigen, and a fungal antigen. In some embodiments, the microbial antigen comprises a purified or recombinant surface protein. In some embodiments, the microbial antigen comprises an inactivated, whole virus.

In some embodiments, the composition does not comprise LPS or MPLA. In some embodiments, the composition does not comprise oxPAPC or a species of oxPAPC. In some embodiments, the composition does not comprise HOdiA-PC, KOdiA-PC, HOOA-PC, KOOA-PC, and/or PGPC. In some embodiments, the composition does not comprise isolated mRNA. In some embodiments, the composition does not comprise a surfactant (e.g., a poloxamer). In some embodiments, the composition does not comprise Poloxamer 407 (KP407), Poloxamer 188 (KP188), and/or Pluronic P123 (P123).

In some embodiments, the composition further comprises an adjuvant, wherein the adjuvant comprises an aluminum salt adjuvant, a squalene-in-water emulsion, a saponin, or combinations thereof.

In some embodiments, the present disclosure provides a pharmaceutical formulation comprising the composition of any of the preceding aspects and a pharmaceutically acceptable excipient. In some embodiments, the formulation does not comprise a surfactant (e.g., a poloxamer). In some embodiments, the formulation does not comprise Poloxamer 407 (KP407), Poloxamer 188 (KP188), and/or Pluronic P123 (P123).

In other aspects, the present disclosure provides a method for production of hyperactivated dendritic cells, the method comprising contacting the dendritic cells with an effective amount of a composition comprising an isolated lysophosphatidylcholine (LPC) with a single C13-C22 acyl chain or a C13-C24 acyl chain, at least one further lipid, and a TLR7/8 agonist for production of hyperactivated dendritic cells, wherein the hyperactivated dendritic cells secrete IL-1beta without undergoing pyroptosis, and the LPC and the at least one further lipid are part of a lipid nanoparticle (LNP). In some embodiments, the at least one further lipid is selected from the group consisting of an ionizable lipid, a cationic lipid, a further phospholipid, a pegylated lipid, a structural lipid, and mixtures thereof. In some embodiments, the dendritic cells are contacted ex vivo with the composition or pharmaceutical formulation of any one of the preceding embodiments. In other embodiments, the dendritic cells are contacted in vivo with the pharmaceutical formulation comprising the composition of any one of the preceding embodiments. In some aspects, the present disclosure provides a pharmaceutical formulation comprising a plurality of the hyperactivated dendritic cells produced by the preceding embodiments, and a pharmaceutically acceptable excipient. In some embodiments, the plurality comprises at least 10³, 10⁴, 10⁵, 10⁶, 10⁷ or 10⁸ hyperactivated DCs.

In other aspects, the present disclosure provides a composition comprising an isolated lysophosphatidylcholine (LPC) with a single acyl chain, at least one further lipid, and a pathogen recognition receptor (PRR) agonist, wherein the acyl chain is a C13-C22 acyl chain or a C13-C24 acyl chain, and the LPC and the at least one further lipid are part of a lipid nanoparticle (LNP). In some embodiments, the at least one further lipid is selected from the group consisting of an ionizable lipid, a cationic lipid, a further phospholipid, a pegylated lipid, a structural lipid, and mixtures thereof. In some embodiments, the PRR agonist is an agonist of a toll-like receptor (TLR), a NOD-like receptor (NLR), a RIG-I-like receptor (RLR), or a C-type lectin receptor (CLR). In some embodiments, the PRR agonist is an agonist of a cytosolic DNA sensor (CDS) or a stimulator of IFN genes (STING). In some embodiments, the PRR agonist comprises a TLR7/8 agonist. In some embodiments, the composition further comprises an antigen and/or dendritic cells.

In some embodiments of the preceding aspects, the acyl chain is a C21-C24 acyl chain. In some embodiments, the acyl chain is a C22 acyl chain. In some embodiments, the acyl chain is fully saturated. In some embodiments, the LPC comprises 1-behenoyl-2-hydroxy-sn-glycero-3-phosphocholine [LPC(22:0)].

In some embodiments of the preceding aspects, the TLR7/8 agonist is a small molecule with a molecule weight of 900 daltons or less. In some embodiments, the TLR7/8 agonist comprises an imidazoquinoline compound. In some embodiments, the TLR7/8 agonist comprises resiquimod (R848). In some embodiments, the LPC comprises LPC(22:0), and the TLR7/8 agonist comprises resiquimod (R848).

The present disclosure further provides compositions for hyperactivation of human dendritic cells, comprising an isolated lysophosphatidylcholine (LPC) compound with a single acyl chain, at least one further lipid, and a pathogen recognition receptor (PRR) agonist, wherein the acyl chain is C22 acyl chain, the LPC and the at least one further lipid are part of a lipid nanoparticle (LNP), and the composition is effective for achieving a higher level of dendritic cell hyperactivation than a comparator composition comprising a comparator compound in place of the LPC. In some embodiments, the at least one further lipid is selected from the group consisting of an ionizable lipid, a cationic lipid, a further phospholipid, a pegylated lipid, a structural lipid, and mixtures thereof. In some embodiments, the hyperactivation occurs in vitro or ex vivo. In other embodiments, the hyperactivation occurs in vivo. In some embodiments, the higher level of dendritic cell hyperactivation comprises induction of IL-1beta secretion from the mammalian (e.g., human) dendritic cells in vitro at a level that is at least 2, 3 or 4 fold higher when contacted with the composition comprising the LPC and the PRR agonist than when contacted with the comparator composition comprising the comparator compound and the PRR agonist, wherein the PRR agonist is LPS. In some embodiments, the concentration of the LPC and the concentration of the comparator compound are the same concentration, optionally in a range of from about 10 μM to about 80 μM, and the LPS is present at a concentration of 1 μg/ml in both the composition and the comparator composition. In some embodiments, the higher level of dendritic cell hyperactivation comprises a lipid activity index for IL-1beta secretion from the mammalian (e.g., human) dendritic cells for the composition comprising the LPC and the PRR agonist that is at least 4, 5 or 6 fold higher in activity units than that of the comparator composition comprising the comparator compound and the PRR agonist. In some embodiments, the comparator compound is PGPC.

### BRIEF DESCRIPTION OF THE DRAWINGS

FIG. 1A-1B show IL-1β secretion by canine peripheral blood mononuclear cells (PBMCs) two days post-activation with the indicated stimuli, shown as total concentration (FIG. 1A) and fold change per donor relative to R848 alone (FIG. 1B), respectively. The results demonstrate that 22:0 LYSO PC, when combined with R848, is capable of stimulating canine PBMCs to secrete IL-1β at levels comparable or higher than DAMPs such as PGPC or LPS and Alum. FIG. 1C shows the relative viability of canine PBMCs two days post-activation with the indicated stimuli. The results demonstrate that canine PBMCs remain viable after treatment with 22:0 LYSO PC.

FIG. 2A-2B show IL-1β secretion by human PBMCs two days post-activation with the indicated stimuli, shown as total concentration (FIG. 2A) and fold change per donor relative to R848 alone (FIG. 2B). The results demonstrate that 22:0 LYSO PC, when combined with R848, is capable of stimulating human PBMCs to secrete IL-1β at levels comparable or higher than DAMPs such as PGPC or LPS and Alum. FIG. 2C shows the relative viability of human PBMCs two days post-activation with the indicated stimuli. The results demonstrate that human PBMCs remain viable after treatment with 22:0 LYSO PC.

FIG. 3A-3B show IFNγ (FIG. 3A) and TNFα (FIG. 3B) secretion by human PBMCs two days-post activation with the indicated stimuli, shown as fold change per donor relative to R848 alone. The results demonstrate that 22:0 LYSO PC, when combined with R848, is capable of stimulating human PBMCs to secrete other immunostimulatory cytokines at levels comparable or higher than DAMPs such as PGPC or LPS and Alum.

FIG. 4 shows that inclusion of 22:0 Lyso PC at varying concentrations in lipid nanoparticles (LNPs) does not impact the size of the resulting LNP. In this plot, the vehicle control for LNP 2 and LNP 3 were the same.

FIG. 5A shows the quantification of inclusion of 22:0 Lyso PC in GenVoy LNPs. FIG. 5B shows the quantification of inclusion of 22:0 Lyso PC in LNPs containing an ionizable lipid. FIG. 5C shows the quantification of inclusion of 22:0 Lyso PC in LNPs lacking an ionizable lipid. FIG. 5D shows that increasing 22:0 Lyso PC input results in increased loading of 22:0 Lyso PC in LNPs.

FIG. 6A shows viability of monocyte-derived dendritic cells (moDCs) cultured in the presence or absence of LNPs and in the presence or absence of a pathogen-associated molecular pattern-containing molecule (PAMP). FIG. 6B shows IL-1β secretion by moDCs cultured in the presence or absence of LNPs and in the presence or absence of a PAMP. The PAMP utilized in the assays of FIG. 6A-6B was resiquimod (R848), which is a the TLR7/8 agonist. When present, 22:0 Lyso PC was included at a concentration of 82.5 μM.

FIG. 7A shows that 22:0 Lyso PC in PBS is large in diameter with a large range in particle size. FIG. 7B shows that 22:0 Lyso PC loaded into LNPs results in a much smaller particle size, with increased uniformity.

FIG. 8A-D show that 22:0 Lyso PC LNPs induce hyperactivation of murine bone marrow-derived dendritic cells (BMDCs). FIG. 8A shows viability of BMDCs 48 hours post-treatment with various formulations, as measured using the Cell Titer Glow assay. Data presented are relative to R848 treated cells. FIG. 8B shows IL-6 secretion and FIG. 8C shows IL-1β secretion by BMDCs 48 hours post-treatment with various formulations, as measured by ELISA. Means and SD are shown and are representative of three replicates from one experiment.

FIG. 8D shows the absolute number of CD11c+ MHC-II+ DCs in draining lymph nodes that are CFSE+ as measured by flow cytometry. Prior to CFSE staining and injection, BMDCs were treated with various formulations for 24 hours. Unpaired t test was used. Means and SD are shown and are representative of four mice from one experiment.

### DETAILED DESCRIPTION

The present disclosure relates to lipid nanoparticles (LNPs) comprising a lysophosphatidylcholine (LPC) compound and at least one further lipid, and uses thereof in hyperactivating mammalian dendritic cells. The present disclosure also relates to compositions comprising LNPs comprising a LPC and at least one further lipid, wherein the compositions further comprise one or more of a pathogen recognition receptor agonist, an antigen, and mammalian dendritic cells, as well as methods for production and use of the compositions. In some embodiments, the dendritic cells are human dendritic cells. In other embodiments, the dendritic cells are non-human dendritic cells. In some embodiments, the non-human dendritic cells are not rodent dendritic cells. In some embodiments, the at least one further lipid is selected from the group consisting of an ionizable lipid, a cationic lipid, a further phospholipid, a pegylated lipid, a structural lipid, and mixtures thereof.

In some embodiments of the present disclosure, the LNPs of the compositions are enriched in particles with lipid bilayer (liposomes) relative to particles with a single lipid layer (micelle). Specifically, in some embodiments, the LNPs comprise liposomes, and little to no micelles.

### General Techniques and Definitions

The practice of the present disclosure will employ, unless otherwise indicated, conventional techniques of molecular biology (including recombinant techniques), microbiology, cell biology, biochemistry and immunology, which are within the skill of the art.

As used herein and in the appended claims, the singular forms “a”, “an”, and “the” include plural references unless indicated otherwise. For example, “an” excipient includes one or more excipients.

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments.

The term “about” as used herein in reference to a value, encompasses from 90% to 110% of that value (e.g., a molecular weight of about 900 daltons, refers to a molecular weight of from 810 daltons to 990 daltons).

An “effective amount” or a “sufficient amount” of a substance is that amount sufficient to effect beneficial or desired results, including clinical results, and, as such, an “effective amount” depends upon the context in which it is being applied. For instance, in the context of administering an immunogenic composition, an effective amount contains sufficient antigen, and one or both of a lysophosphatidylcholine (LPC) compound and a PRR agonist, to stimulate an immune response against the antigen (e.g., antigen-reactive antibody and/or cellular immune response).

The terms “individual” and “subject” refer to mammals. “Mammals” include, but are not limited to, humans, non-human primates (e.g., monkeys), farm animals, sport animals, rodents (e.g., mice and rats), and pets (e.g., dogs and cats). In some embodiments, the subject is a human patient, such as a human patient suffering from cancer and/or an infectious disease.

The term “dose” as used herein in reference to an immunogenic composition refers to a measured portion of the immunogenic composition taken by (administered to or received by) a subject at any one time.

The terms “isolated” and “purified” as used herein refers to a material that is removed from at least one component with which it is otherwise associated during production of the material (e.g., removed from its original environment). As an example, when used in reference to an LPC, an isolated LPC is at least 90%, 95%, 96%, 97%, 98% or 99% pure as determined by thin layer chromatography, or gas chromatography. As a further example, when used in reference to a recombinant protein, an isolated protein refers to a protein that has been removed from the culture medium of the host cell that produced the protein. Additionally, when used in reference to a synthesized compound, an isolated compound or a purified compound has been removed from the reaction mixture in which it was synthesized.

The terms “pharmaceutical formulation” and “pharmaceutical composition” refer to preparations that are in such form as to permit the biological activity of the active ingredient to be effective, and that contain no additional components that are unacceptably toxic to an individual to which the formulation or composition would be administered. Such formulations or compositions are intended to be sterile.

“Excipients” as used herein include pharmaceutically acceptable excipients, carriers, vehicles or stabilizers that are nontoxic to the cell or mammal being exposed thereto at the dosages and concentrations employed. Often the physiologically acceptable excipient is an aqueous pH buffered solution.

The term “antigen” refers to a substance that is recognized and bound specifically by an antibody or by a T cell antigen receptor. Antigens can include peptides, polypeptides, proteins, glycoproteins, polysaccharides, complex carbohydrates, sugars, gangliosides, lipids and phospholipids; portions thereof and combinations thereof. Antigens when present in the compositions of the present disclosure can be synthetic or isolated from nature. Antigens suitable for administration in the methods of the present disclosure include any molecule capable of eliciting an antigen-specific B cell or T cell response. Haptens are included within the scope of “antigen.” A “hapten” is a low molecular weight compound that is not immunogenic by itself but is rendered immunogenic when conjugated with a generally larger immunogenic molecule (carrier).

“Polypeptide antigens” can include purified native peptides, synthetic peptides, recombinant peptides, crude peptide extracts, or peptides in a partially purified or unpurified active state (such as peptides that are part of attenuated or inactivated viruses, microorganisms or cells), or fragments of such peptides. Polypeptide antigens are preferably at least eight amino acid residues in length.

The term “agonist” is used in the broadest sense and includes any molecule that activates signaling through a receptor. In some embodiments, the agonist binds to the receptor. For instance, a TLR8 agonist binds to a TLR8 receptor and activates a TLR8-signaling pathway.

“Alkyl” refers to monovalent saturated aliphatic hydrocarbyl groups. Cx alkyl refers to an alkyl group having x number of carbon atoms. Cx-Cy alkyl or Cx-y alkyl refers to an alkyl group having between x number and y number of carbon atoms, inclusive.

“Alkylene” refers to divalent saturated aliphatic hydrocarbyl groups.

“Alkenyl” refers to monovalent hydrocarbyl groups having at least one double bond (>C═C<). Cx alkenyl refers to an alkenyl group having x number of carbon atoms. Cx-Cy alkenyl or Cx-y alkenyl refers to an alkenyl group having between x number and y number of carbon atoms, inclusive.

“Stimulation” of a response or parameter includes eliciting and/or enhancing that response or parameter when compared to otherwise same conditions except for a parameter of interest, or alternatively, as compared to another condition (e.g., increase in TLR-signaling in the presence of a TLR agonist as compared to the absence of the TLR agonist). For example, “stimulation” of an immune response means an increase in the response. Depending upon the parameter measured, the increase may be from 2-fold to 2,000-fold, or from 5-fold to 500-fold or over, or from 2, 5, 10, 50, or 100-fold to 500, 1,000, 2,000, 5,000, or 10,000-fold.

Conversely, “inhibition” of a response or parameter includes reducing and/or repressing that response or parameter when compared to otherwise same conditions except for a parameter of interest, or alternatively, as compared to another condition (e.g., decrease in abnormal cell proliferation after administration of a composition comprising a LPC compound and one or more of a pathogen recognition receptor agonist, an antigen, and human dendritic cells, as compared to the administration of a placebo composition or no treatment). For example, “inhibition” of an immune response means a decrease in the response. Depending upon the parameter measured, the decrease may be from 2-fold to 2,000-fold, or from 5-fold to 500-fold or over, or from 2, 5, 10, 50, or 100-fold to 500, 1,000, 2,000, 5,000, or 10,000-fold.

The relative terms “higher” and “lower” refer to a measurable increase or decrease, respectively, in a response or parameter when compared to otherwise same conditions except for a parameter of interest, or alternatively, as compared to another condition. For instance, a “higher level of DC hyperactivation” refers to a level of DC hyperactivation as a consequence of a treatment condition (comprising a LPC compound of the present disclosure) that is at least 2, 3, 4, 5, 6, 7, 8, 9, or 10-fold above a level of DC hyperactivation as a consequence of a control condition (e.g., no LPC, PGPC, oxPAPC, etc.). Likewise, a “lower level of DC hyperactivation” refers to a level of DC hyperactivation as a consequence of a treatment condition (comprising a LPC compound of the present disclosure) that is at least 2, 3, 4, 5, 6, 7, 8, 9, or 10-fold below a level of DC hyperactivation as a consequence of a control condition (e.g., no LPC, PGPC, oxPAPC, etc.). In some embodiments, the control condition comprises a comparator compound in the place of the LPC of the treatment condition.

As used herein the term “immunization” refers to a process that increases a mammalian subject's reaction to antigen and therefore improves its ability to resist or overcome infection and/or resist disease.

The term “vaccination” as used herein refers to the introduction of vaccine into a body of a mammalian subject.

“Adjuvant” refers to a substance which, when added to a composition comprising an antigen, enhances or potentiates an immune response to the antigen in the mammalian recipient upon exposure.

The terms “treating” or “treatment” of a disease refer to executing a protocol, which may include administering one or more therapeutic agents to an individual (human or otherwise), in an effort to obtain beneficial or desired results in the individual, including clinical results. Beneficial or desired clinical results include, but are not limited to, alleviation or amelioration of one or more signs or symptoms of a disease, diminishment of extent of disease, stabilized (i.e., not worsening) state of disease, preventing spread of disease, delay or slowing of disease progression, amelioration or palliation of the disease state, and remission (whether partial or total). “Treatment” also can mean prolonging survival as compared to expected survival of an individual not receiving treatment. Further, “treating” and “treatment” may occur by administration of one dose of a therapeutic agent or therapeutic agents, or may occur upon administration of a series of doses of a therapeutic agent or therapeutic agents. “Treating” or “treatment” does not require complete alleviation of signs or symptoms, and does not require a cure, and specifically includes protocols that have only a palliative effect on the individual. “Palliating” a disease or disorder means that the extent and/or undesirable clinical manifestations of the disease or disorder are lessened and/or time course of progression of the disease or disorder is slowed, as compared to the expected untreated outcome.

### I. Lysophosphatidylcholine Compounds

A “lysophosphatidylcholine” (LPC) or “lysophosphatidylcholine molecule” refers to a glycerol molecule bearing one phosphocholine group on a hydroxyl group of the glycerol and bearing one acyl group on one of the other two hydroxyl groups of the glycerol. The remaining hydroxyl group is unsubstituted.

In some embodiments, the isolated lysophosphatidylcholine (LPC) with a single acyl chain is of the form:

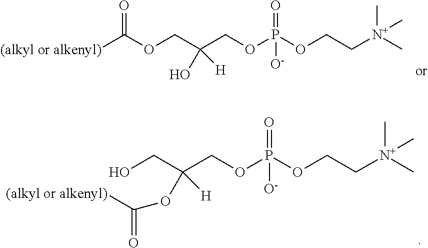

In some embodiments, the isolated lysophosphatidylcholine (LPC) with a single acyl chain is of the form:

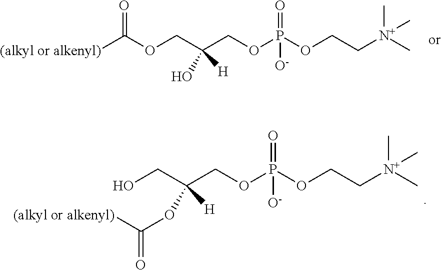

The alkyl or alkenyl chain, together with the carbonyl carbon, forms an acyl chain which is one carbon atom longer than the alkyl or alkenyl chain. For example, a (C23 alkyl)-C(═O)— group forms a C24 acyl chain. Thus, when the group “(alkyl or alkylene)” is a C12-C23 alkyl group (such as a C12-C19 alkyl group or a C20-C23 alkyl group), the (C12-C23 alkyl-C(═O)— group forms a C13-C24 acyl chain (such as a C13-C20 acyl chain or a C21-C24 acyl chain). When the group “(alkyl or alkylene)” is a C12-C23 alkenyl group (such as a C12-C19 alkenyl group or a C20-C23 alkenyl group), the (C12-C23 alkenyl-C(═O)— group forms a C13-C24 acyl chain (such as a C13-C20 acyl chain or a C21-C24 acyl chain). Acyl chains can be referred to as saturated acyl or unsaturated acyl to distinguish between alkyl-containing and alkenyl-containing acyl groups. Standard delta notation or omega notation can be used to indicate the position of one or more double bonds in an unsaturated acyl chain.

Lysophosphatidylcholine (LPC) compounds of the present disclosure have a single acyl chain in which the acyl chain is a C13-C22 acyl chain or a C13-C24 acyl chain. In some embodiments, the acyl chain is a C18-C22 acyl chain or a C21-C24 acyl chain. In some preferred embodiments, the acyl chain is a C22 acyl chain. Names and structures of exemplary LPC compounds for inclusion in LNPs of the present disclosure, as well as their Chemical Abstract Service (CAS) Registry Numbers are listed as Compounds #30-#43, optionally #30-#42 of Table I of International Application No. PCT/US2022/071664, which is incorporated herein by reference. Several methods are known for synthesis of lysophospholipids (see, e.g., D′Arrigo et al, “Synthesis of lysophospholipids,” Molecules, 15 (3): 1354-77, 2010; and Yang et al., “Lysophosphatidylcholine synthesis by lipase-catalyzed ethanolysis,” J Oleo Sci., 64 (4): 443-7, 2015, and the references cited therein). Additionally, many lysophospholipids are commercially available.

### II. Pathogen Recognition Receptor Agonists

Compositions and methods of the present disclosure may further comprise a pathogen recognition receptor (PRR) agonist. In some embodiments, the PRR agonist comprises an agonist of a toll-like receptor (TLR), a NOD-like receptor (NLR), a RIG-I-like receptor (RLR), or a C-type lectin receptor (CLR). In other embodiments, the PRR agonist comprises a cytosolic DNA sensor (CDS) or a stimulator of IFN genes (STING). In some embodiments, the PRR agonist comprises a TLR7/8 agonist.

### A. TLR7/8 Agonists

The term “TLR7/8 agonist” as used herein refers to an agonist of TLR7 and/or TLR8. In one aspect, the TLR7/8 agonist is a TLR7 agonist. In another aspect, the TLR7/8 agonist is a TLR8 agonist. In a further aspect, the TLR7/8 agonist is an agonist of both TLR7 and TLR8. TLR7/8 agonists of the present disclosure are suitable for hyperactivating human dendritic cells in the presence of LPC.

In some aspects, the TLR7/8 agonist is a small molecule. In some embodiments, the TLR7/8 agonist is a small molecule with a molecule weight of 900 daltons or less, or a salt thereof. That is, the small molecule TLR7/8 agonist is not a large molecule like a recombinant protein or a synthetic oligonucleotide, which is regulatable by the U.S. FDA's Center for Biologics Evaluation and Research. Rather the small molecule TLR7/8 agonist is regulatable by the FDA's Center for Drug Evaluation and Research. In some embodiments, the small molecule has a molecule weight of from about 90 to about 900 daltons. In some embodiments, the TLR7/8 agonist comprises an imidazoquinoline compound. In some preferred embodiments, the TLR7/8 agonist comprises resiquimod (R848).

### B. Other PRR Agonists

In some aspects, the pathogen recognition receptor (PRR) agonist comprises a toll-like receptor (TLR) agonist with the proviso that the TLR agonist does not comprise a TLR7/8 agonist. In some embodiments, the TLR agonist comprises an agonist of one or more of TLR2, TLR3, TLR4, TLR5, TLR9 and TLR13. In some embodiments, the PRR agonist is a TLR2/6 agonist, such as Pam2CSK4. In other embodiments, the TLR agonist is a TLR4 agonist such as monophosphoryl lipid A (MPLA). However, in preferred embodiments, the TLR agonist is not an agonist of TLR2, TLR4 and/or TLR9. For instance, in preferred embodiments, the TLR9 agonist is not a TLR4 ligand such as LPS (endotoxin).

In other aspects, the PRR agonist comprises a NOD-like receptor (NLR) agonist. In further aspects, the PRR agonist comprises a RIG-I-like receptor (RLR) agonist. In additional aspects, the PRR agonist comprises a C-type lectin receptor (CLR) agonist. In still further aspects, the PRR agonist comprises a CDS agonist or a STING agonist.

### III. Antigens

Compositions and methods of the present disclosure may further comprise an antigen. In some embodiments, the antigen comprises a proteinaceous antigen. The terms “polypeptide” and “protein” are used interchangeably herein to refer to proteinaceous antigens that comprise peptide chains that are at least 8 amino acids in length. In some embodiments, the proteinaceous antigen is from 8 to 1800 amino acids, 9 to 1000 amino acids, or 10 to 100 amino acids in length. In some embodiments, the antigen comprises a synthetic protein or a recombinant protein. In other embodiments, the antigen comprises a protein purified from a biological sample. The polypeptide may be post-translationally modified such as by phosphorylation, hydroxylation, sulfonation, palmitoylation, and/or glycosylation.

In some embodiments, the antigen is a tumor antigen that comprises the amino acid sequence of at least one full length protein or fragment thereof. In some embodiments, the tumor antigen comprises an amino acid sequence or fragment thereof from an oncoprotein. In some embodiments, the mammalian antigen is a neoantigen or encoded by a gene comprising a mutation relative to the gene present in normal cells from a mammalian subject. Neoantigens are thought to be particularly useful in enabling T cells to distinguish between cancer cells and non-cancer cells (see, e.g., Schumacher and Schreiber, Science, 348:69-74, 2015). In other embodiments, the tumor antigen comprises a viral antigen, such as an antigen of a cancer-causing virus.

In some embodiments, the tumor antigen is a fusion protein comprising two or more polypeptides, wherein each polypeptide comprises an amino acid sequence from a different tumor antigen or non-contiguous amino acid sequences from the same tumor antigen. In some of these embodiments, the fusion protein comprises a first polypeptide and a second polypeptide, wherein each polypeptide comprises non-contiguous amino acid sequences from the same tumor antigen.

In some embodiments, the antigen is a microbial antigen. In some embodiments, the microbial antigen comprises a viral antigen, a bacterial antigen, a protozoan antigen, a fungal antigen, or combinations thereof. In some embodiments, the microbial antigen comprises a surface protein or other antigenic subunit of a microbe. In other embodiments, the microbial antigen comprises an inactivated or attenuated microbe. For instance, the microbial antigen may comprise an inactivated virus, such as a chemically or genetically-inactivated virus. Alternatively, the microbial antigen may comprise a virus-like particle.

In some embodiments, the antigen may be present in a biological sample obtained from an individual, such as a human patient. For instance, the antigen may comprise cancer cells. In another aspect, the antigen may comprise microbially-infected cells, such as virally-infected cells.

### IV. Dendritic Cells

Compositions and methods of the present disclosure may further comprise dendritic cells (DCs), which are antigen presenting cells that are thought to bridge the innate and adaptive immune systems of mammals. In preferred embodiments, the DCs are subset-1 conventional DCs (cDC1s, previously referred to as myeloid DC1s), as opposed to plasmacytoid DCs (pDCs).

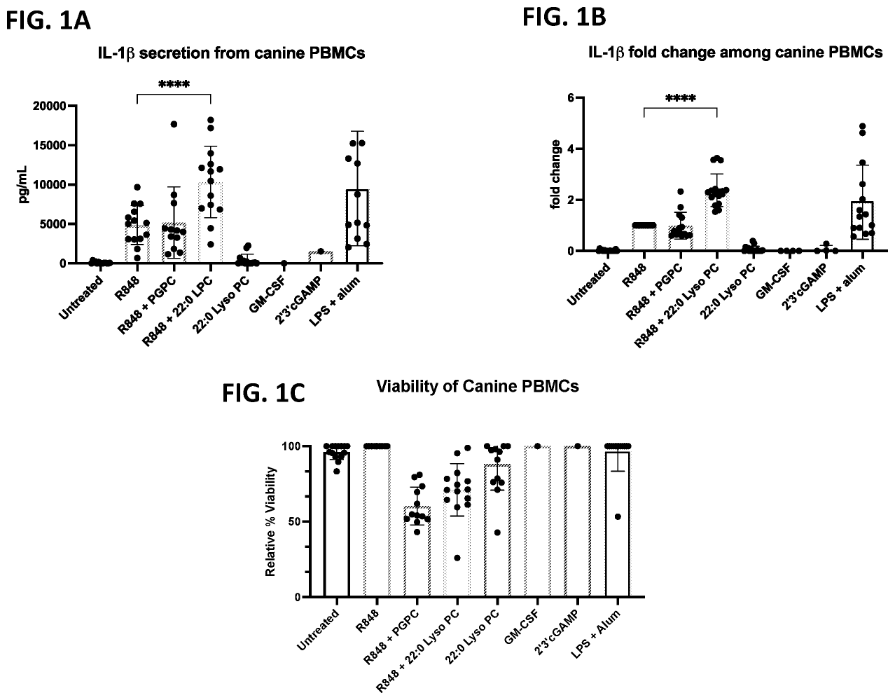

In some embodiments, the DCs are hyperactive DCs that express high levels of CD40 and IL-12p70. As used herein, the term “hyperactive dendritic cells” refer to a cell state in which DCs are able to secrete IL-1β while maintaining cellular viability (e.g., without undergoing pyroptosis). In this way, hyperactivated dendritic cells are able to stimulate robust T cell immunity (FIG. 1), which apparently combines the benefits of activated and pyroptotic dendritic cells (Zhivaki et al., Cell Reports, 33 (7), 2020, 108381).

### V. Further Lipids

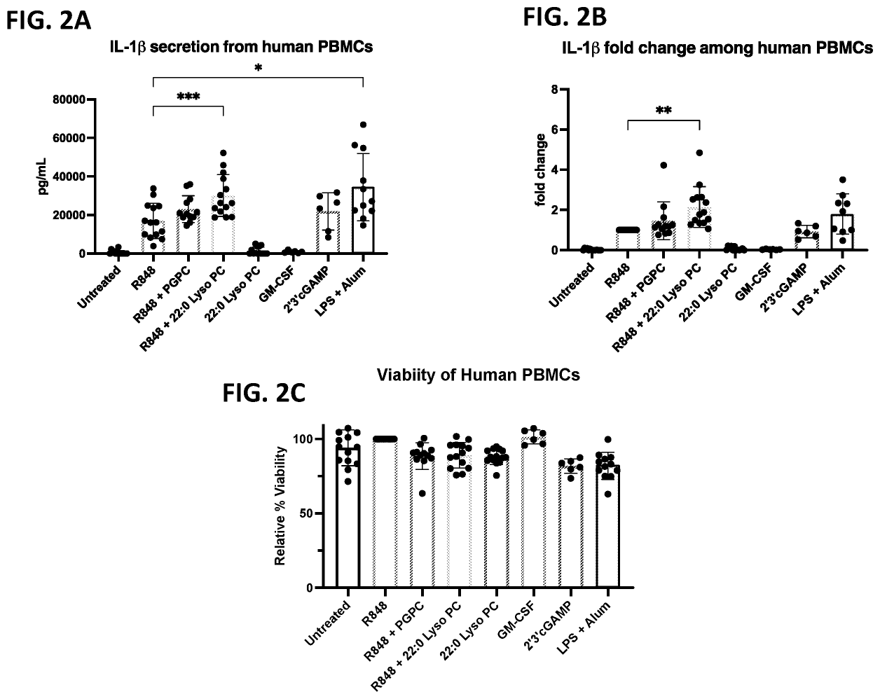

Compositions and methods of the present disclosure comprise at least one further lipid, wherein the LPC and the at least one further lipid are part of a lipid nanoparticle (LNP). In some embodiments, the at least one further lipid comprises an ionizable lipid, a cationic lipid, a further phospholipid, a pegylated lipid, a structural lipid, or a mixture thereof. In some embodiments, the LNP comprises a first phospholipid (lysophosphatidylcholine with a single C13-C24 acyl chain [LPC: C13-C24]), an ionizable lipid, a second phospholipid, a pegylated lipid, and a structural lipid. Structures of further lipids suitable for use in the compositions and methods of the present disclosure are shown below (reproduced from FIG. 2 of Hou et al., Nature Review Materials, 6:1078-1094, 2021).

In some embodiments, the at least one further lipid comprises one or both of a further phospholipid and a structural lipid, optionally wherein the further phospholipid comprises 1,2-distearoyl-sn-glycero-3-phosphocholine (DSPC), and the structural lipid comprises cholesterol. In some embodiments, the at least one further lipid comprises or further comprises a pegylated lipid, optionally wherein the pegylated lipid comprises polyethylene glycol [PEG] 2000 dimyristoyl glycerol [DMG]. In some embodiments, least one further lipid comprises or further comprises an ionizable lipid, optionally wherein the ionizable lipid comprises (6Z,9Z,28Z,31Z)-heptatriaconta-6,9,28,31-tetraen-19-yl 4-(dimethylamino) butanoate, which is also know as 4-(dimethylamino)-butanoic acid, (10Z,13Z)-1-(9Z,12Z)-9,12-octadecadien-1-yl-10,13-nonadecadien-1-yl ester (DLin-MC3-DMA) or analogs or derivatives thereof.

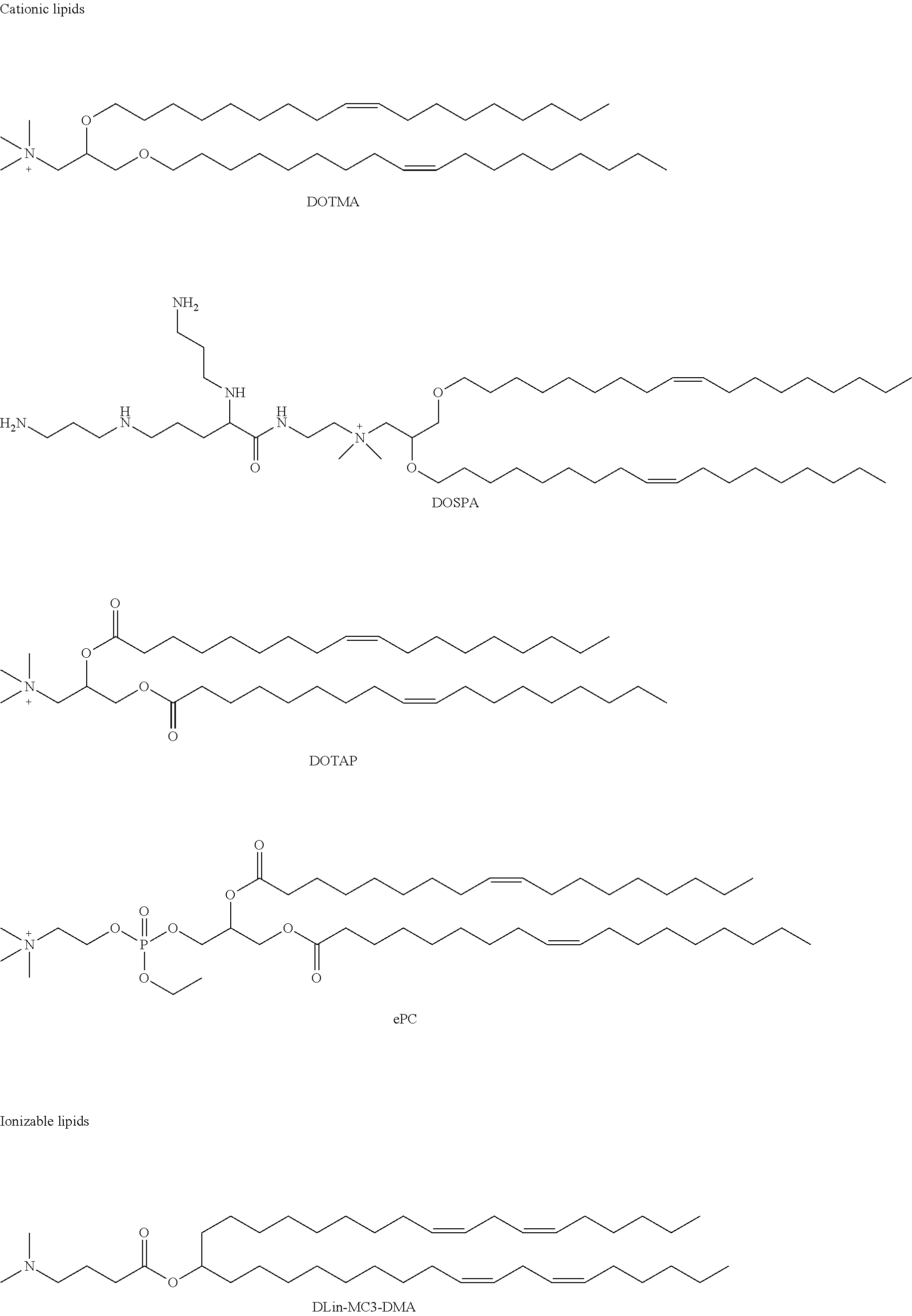

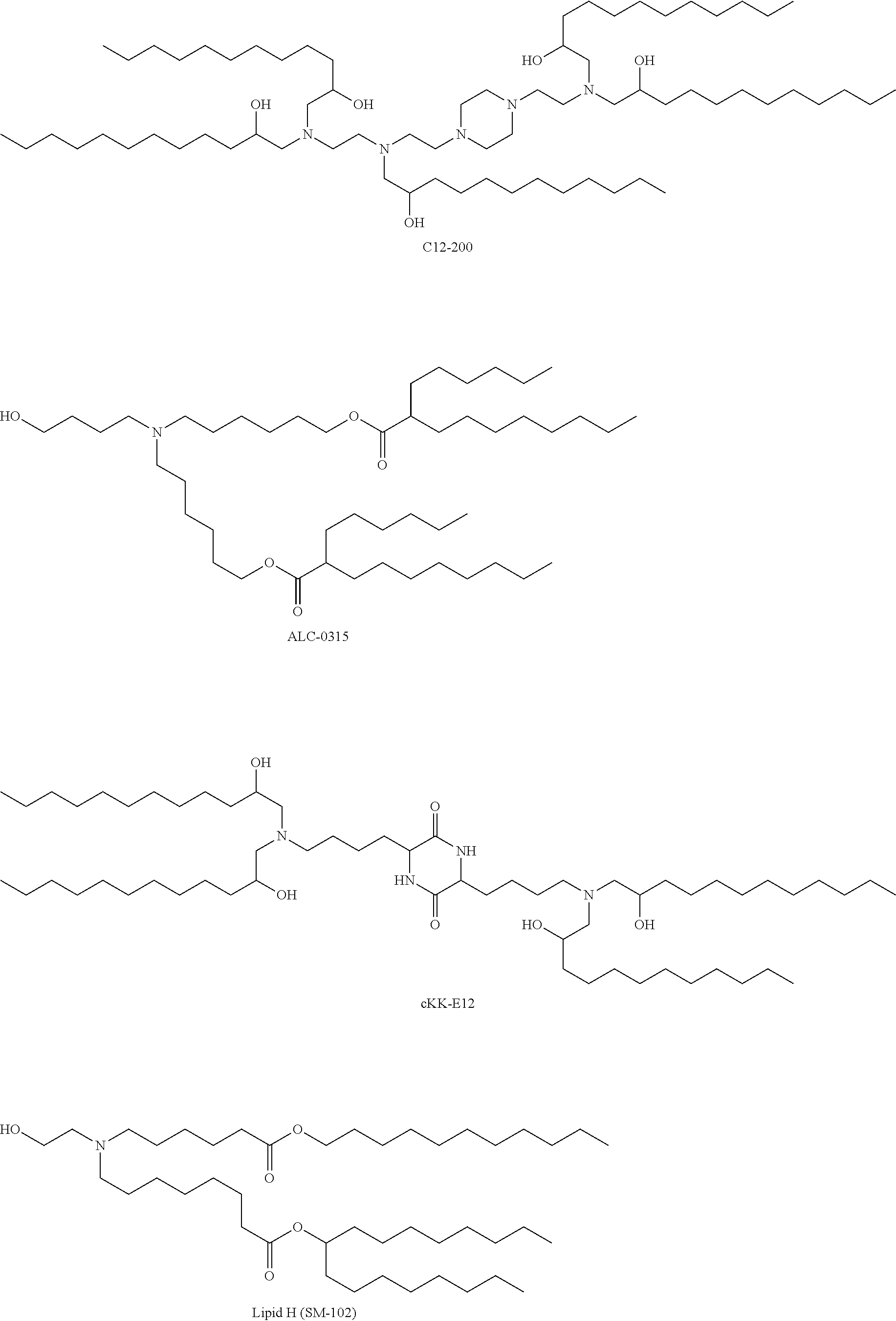

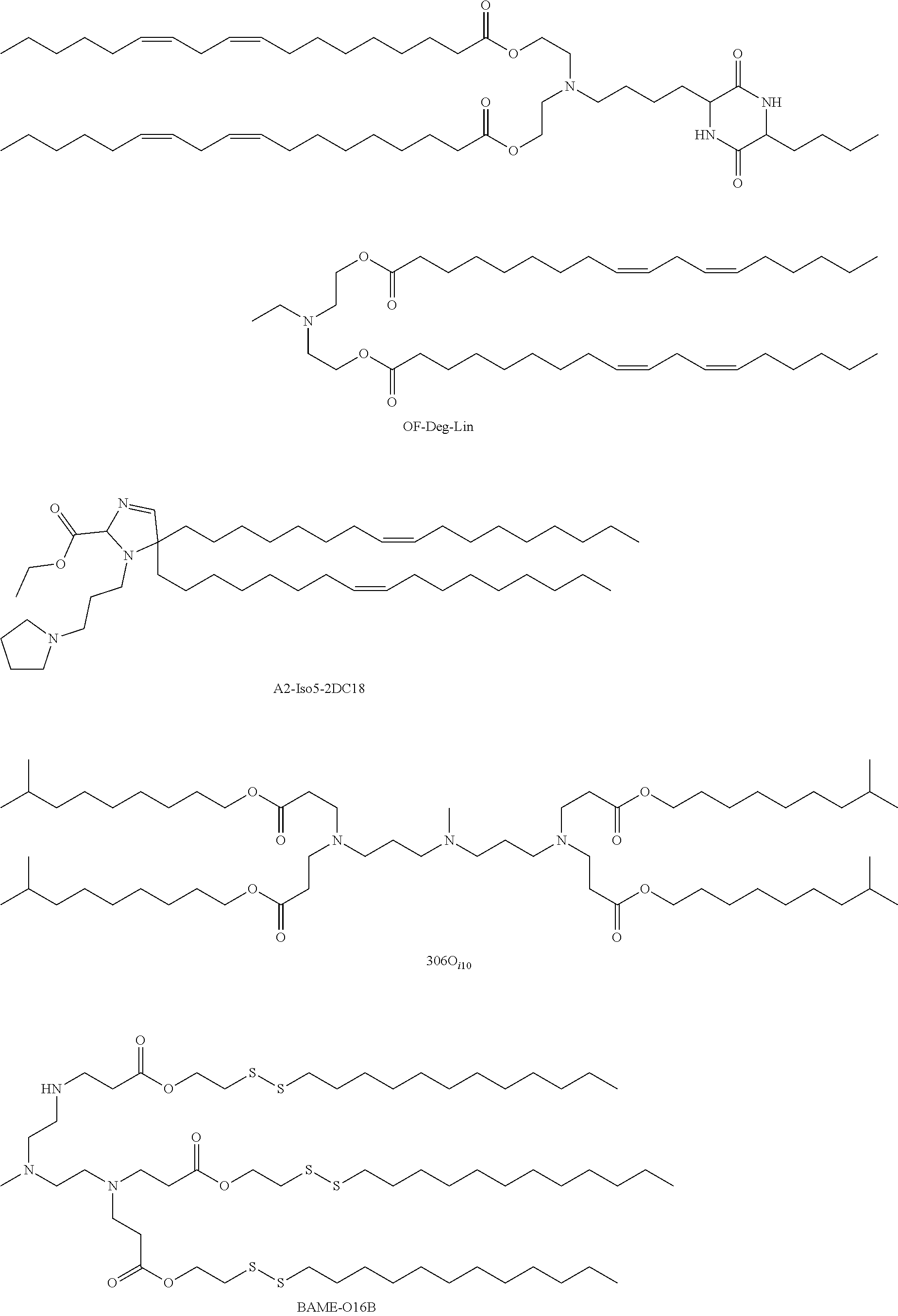

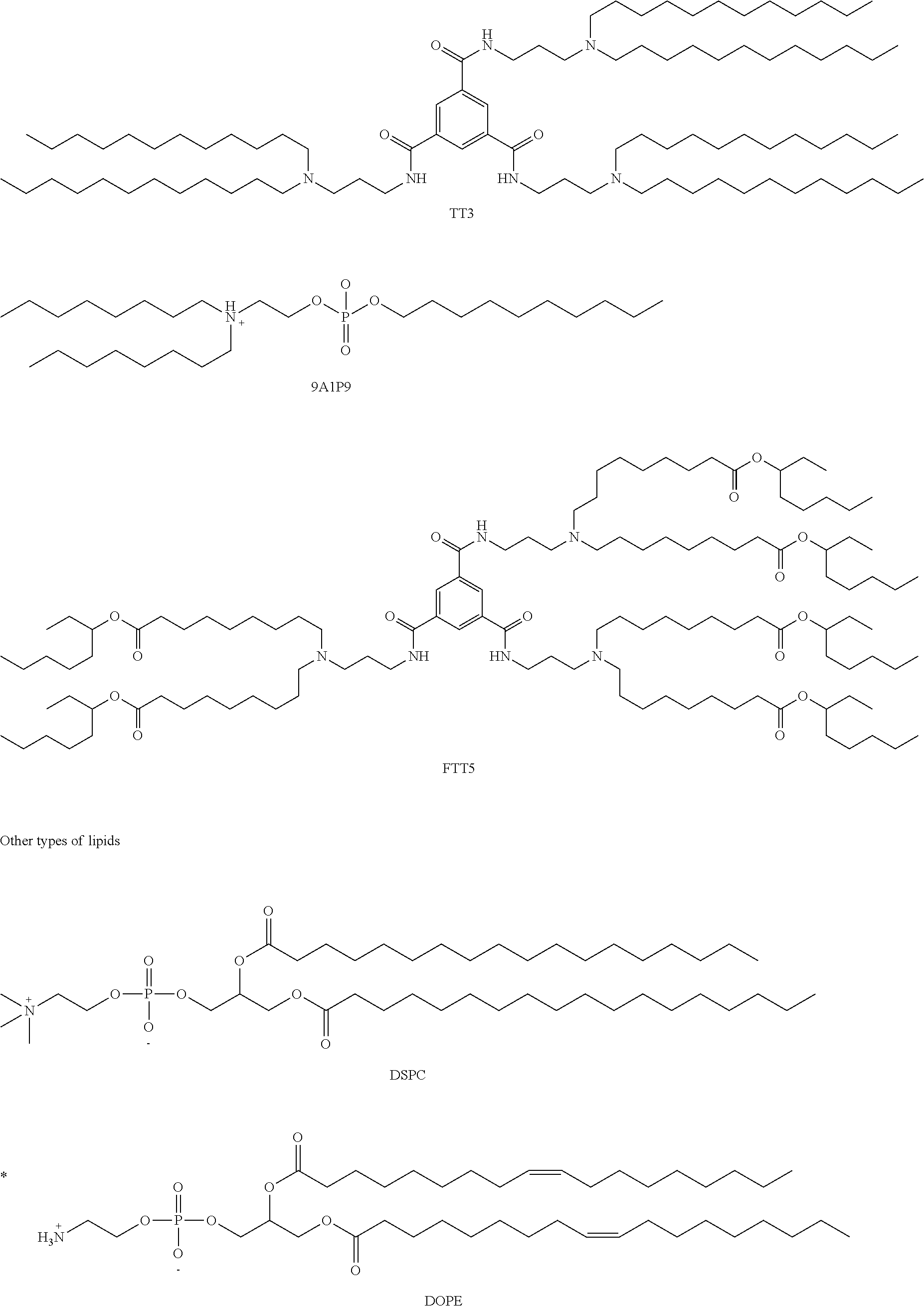

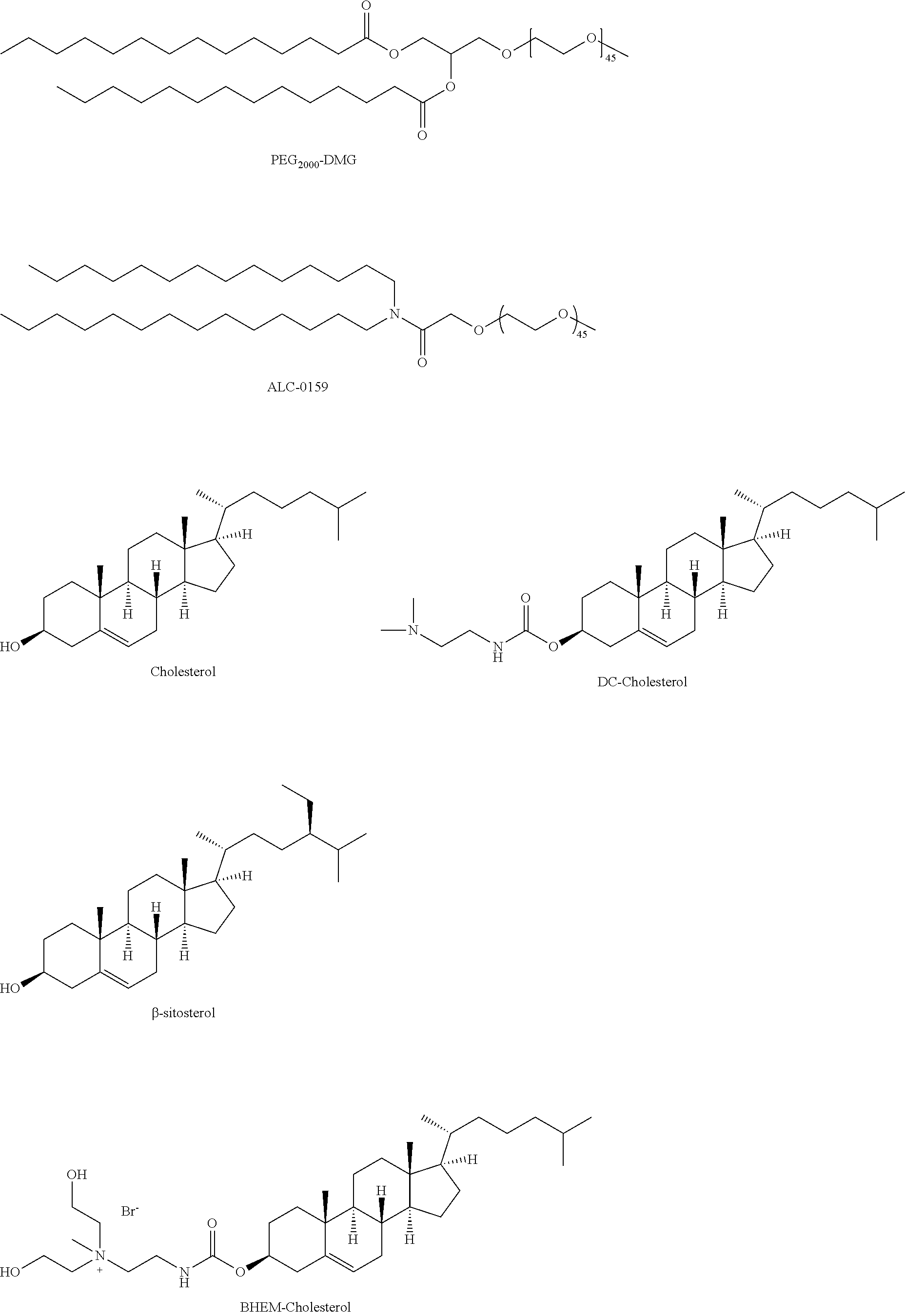

### VI. Pharmaceutical Formulations

Some compositions of the present disclosure are pharmaceutical formulations comprising a pharmaceutically acceptable excipient, and a lipid nanoparticle (LNP) comprising a LPC compound and at least one further lipid. In some embodiments, the at least one further lipid is selected from the group consisting of an ionizable lipid, a cationic lipid, a further phospholipid, a pegylated lipid, a structural lipid, and mixtures thereof. In some embodiments, the pharmaceutical formulations further comprise a PRR agonist, a dendritic cell, an antigen, an adjuvant, or any combination thereof. Pharmaceutical formulations of the present disclosure may be in the form of a solution or a suspension. Alternatively, the pharmaceutical formulations may be a dehydrated solid (e.g., freeze dried or spray dried solid). The pharmaceutical formulations of the present disclosure are preferably sterile, and preferably essentially endotoxin-free. The term “pharmaceutical formulations” is used interchangeably herein with the terms “medicinal product” and “medicament”. In some embodiments, the pharmaceutical formation comprises specific ratios of the various components based on the intended purpose of the formulation.

### A. Excipients

Pharmaceutically acceptable excipients of the present disclosure include for instance, solvents, buffering agents, tonicity adjusting agents, bulking agents, and preservatives (See, e.g., Pramanick et al., Pharma Times, 45:65-77, 2013). In some embodiments, the pharmaceutical formulations may comprise an excipient that functions as one or more of a solvent, a buffering agent, a tonicity adjusting agent, and a bulking agent (e.g., sodium chloride in saline may serve as both an aqueous vehicle and a tonicity adjusting agent).

In some embodiments, the pharmaceutical formulations comprise an aqueous vehicle as a solvent. Suitable vehicles include for instance sterile water, saline solution, phosphate buffered saline, and Ringer's solution. In some embodiments, the composition is isotonic.

The pharmaceutical formulations may comprise a buffering agent. Buffering agents control pH to inhibit degradation of the active agent during processing, storage and optionally reconstitution. Suitable buffers include for instance salts comprising acetate, citrate, phosphate or sulfate. Other suitable buffers include for instance amino acids such as arginine, glycine, histidine, and lysine. The buffering agent may further comprise hydrochloric acid or sodium hydroxide. In some embodiments, the buffering agent maintains the pH of the composition within a range of 6 to 9. In some embodiments, the pH is greater than (lower limit) 6, 7 or 8. In some embodiments, the pH is less than (upper limit) 9, 8, or 7. That is, the pH is in the range of from about 6 to 9 in which the lower limit is less than the upper limit.

The pharmaceutical compositions may comprise a tonicity adjusting agent. Suitable tonicity adjusting agents include for instance dextrose, glycerol, sodium chloride, glycerin and mannitol.

The pharmaceutical formulations may comprise a bulking agent. Bulking agents are particularly useful when the pharmaceutical composition is to be lyophilized before administration. In some embodiments, the bulking agent is a protectant that aids in the stabilization and prevention of degradation of the active agents during freeze or spray drying and/or during storage. Suitable bulking agents are sugars (mono-, di- and polysaccharides) such as sucrose, lactose, trehalose, mannitol, sorbital, glucose and raffinose.

The pharmaceutical formulations may comprise a preservative. Suitable preservatives include for instance antioxidants and antimicrobial agents. However, in preferred embodiments, the pharmaceutical formulation is prepared under sterile conditions and is in a single use container, and thus does not necessitate inclusion of a preservative.

The pharmaceutical and other compositions of the present disclosure are typically devoid of a surfactant (e.g., a poloxamer). In particular, in some embodiments, the pharmaceutical and other compositions are devoid of Poloxamer 407 (KP407), Poloxamer 188 (KP188), and/or Pluronic P123 (P123).

The pharmaceutical formulations of the present disclosure are suitable for parenteral administration. That is the pharmaceutical formulations of the present disclosure are not intended for enteral administration (e.g., not by orally, gastrically, or rectally).

### B. Adjuvants

Pharmaceutically acceptable adjuvants of the present disclosure include for instance, an aluminum salt adjuvant, a squalene-in-water emulsion, a saponin, or combinations thereof. In some embodiments, the adjuvant is an aluminum salt adjuvant selected from the group consisting of amorphous aluminum hydroxyphosphate sulfate, aluminum hydroxide, aluminum phosphate, potassium aluminum sulfate, and combinations thereof. In other embodiments, the adjuvant is a squalene-in-water emulsion such as MF59 or AS03. In other embodiments, the adjuvant is a saponin, such as Quil A or QS-21, as in AS01 or AS02.

### VII. Methods for Production

The present disclosure relates, in some aspects, to methods for preparing hyperactivated dendritic cells, and methods for preparing immunogenic compositions. The immunogenic compositions are suitable for hyperactivation of dendritic cells in vitro, ex vivo, or in vivo.

In one aspect, the present disclosure provides a method for production of hyperactivated dendritic cells (DCs), the method comprising contacting dendritic cells with an effective amount of a composition comprises an isolated lysophosphatidylcholine (LPC) with a single acyl chain, at least one further lipid, and a PRR agonist, for production of hyperactivated dendritic cells, wherein the hyperactivated dendritic cells secrete IL-1beta without undergoing pyroptosis, and the LPC and the at least one further lipid are part of a lipid nanoparticle (LNP). In some embodiments, the at least one further lipid is selected from the group consisting of an ionizable lipid, a cationic lipid, a further phospholipid, a pegylated lipid, a structural lipid, and mixtures thereof. In some embodiments, the DCs are isolated, while in other embodiments, the DCs are present within a biological sample obtained from a mammalian subject, such as a human patient. In some embodiments, the DCs are monocyte-derived DCs, preferably cDC1s.

In specific embodiments, the present disclosure provide a method for production of an immunogenic composition, the method comprising:
a) optionally depleting leukocytes from a suspension of cells prepared from a tumor to obtain a tumor cell-enriched suspension;
b) lysing cells from the tumor cell-enriched suspension to obtain a tumor cell lysate; and
c) contacting the tumor cell lysate with an isolated lysophosphatidylcholine (LPC) having a single acyl chain, at least one further lipid, and a PRR agonist, to obtain the immunogenic composition, wherein the LPC and the at least one further lipid are part of a lipid nanoparticle (LNP). In some embodiments, the at least one further lipid is selected from the group consisting of an ionizable lipid, a cationic lipid, a further phospholipid, a pegylated lipid, a structural lipid, and mixtures thereof. In some embodiments, the leukocytes are depleted from the tumor cell-enriched cell suspension by contacting the tumor cell-enriched suspension with an antibody specific to leukocytes. In some embodiments, the leukocytes are depleted by contacting the tumor cell-enriched suspension with an anti-CD45 antibody. In some embodiments, the cells are lysed by a physical disruption-based cell lysis method, such as, but not limited to, mechanical lysis, liquid homogenization, sonication, freeze-thaw, or manual grinding. In some preferred embodiments, the cells are lysed by one or more freeze-thaw cycles.

In some embodiments of the afore-mentioned methods, the acyl chain of the LPC is a C13-C22 acyl chain or a C13-C24 acyl chain. In some embodiments, the acyl chain of the LPC is a C18-C22 acyl chain or a C18-C24 acyl chain. In some preferred embodiments, the acyl chain is fully saturated. In some preferred embodiments, the acyl chain of the LPC is a C22 acyl chain. In some preferred embodiments, the LPC is 1-behenoyl-2-hydroxy-sn-glycero-3-phosphocholine [LPC(22:0)]. In some embodiments, the PRR agonist is a TLR7/8 agonist. In some preferred embodiments, the TLR7/8 agonist is an imidazoquinoline compound, which in particularly preferred embodiments is resiquimod (R848).

### VIII. Methods of Use

In some aspects, the present disclosure relates to methods of use of any one of the compositions or formulations described herein. In some embodiments, the compositions or formulations comprise an LPC compound, and at least one further lipid, wherein the LPC and the at least one further lipid are part of a lipid nanoparticle (LNP). In some embodiments, the at least one further lipid is selected from the group consisting of an ionizable lipid, a cationic lipid, a further phospholipid, a pegylated lipid, a structural lipid, and mixtures thereof. In some embodiments, the compositions or formulations further comprise a PRR agonist, a dendritic cell, an antigen, an adjuvant, or any combination thereof. The methods of use are suitable for a plurality of uses involving stimulating an immune response. In some embodiments, the methods of use comprise methods of treating cancer. In some embodiments, the methods of use comprise methods of inhibiting abnormal cell proliferation. In some embodiments, the methods of use comprise methods of treating an infectious disease. The methods comprise administering an effective amount of a formulation or a composition described herein to an individual in need thereof to achieve a specific outcome. The individual is a mammalian subject, such as a human patient. In other embodiments, the individual a non-human patient. In some embodiments, the individual is a canine patient. That is in some embodiments, the methods of use involve clinical uses, while in other embodiments the methods of use involve pre-clinical and/or veterinary uses. For preclinical uses, the mammalian subject may be a non-human primate (e.g., monkey or ape) or a rodent (e.g., mouse or rat). For veterinary uses the mammalian subject may be a farm animal (e.g., cow), a sport animal (e.g., horse), a or a pet (e.g., companion animal such as a dog or cat).

### A. Stimulation of an Immune Response

In brief, the present disclosure provides methods of stimulating an immune response in an individual, comprising administering to the individual a composition or formulation described herein in an amount sufficient to stimulate an immune response in the individual. “Stimulating” an immune response (used interchangeably with “eliciting” and immune response), means increasing the immune response, which can arise from eliciting a de novo immune response (e.g., as a consequence of an initial vaccination regimen) or enhancing an existing immune response (e.g., as a consequence of a booster vaccination regimen). In some embodiments, stimulating an immune response comprises one or more of the group consisting of: stimulating cytokine production; stimulating B lymphocyte proliferation; stimulating interferon pathway-associated gene expression; stimulating chemoattractant-associated gene expression; and stimulating dendritic cell DC maturation. Methods for measuring stimulation of an immune response are known in the art.

For instance, the present disclosure provides methods of inducing an antigen-specific immune response in an individual by administering to the individual a composition or formulation described herein in an amount sufficient to induce an antigen-specific immune response in the individual. In preferred embodiments, the composition or formulation comprises the antigen. In some embodiments, the composition or formulation is administered to a tissue of the individual comprising the antigen. The immune response may comprise one or both of an antigen-specific antibody response and an antigen-specific cytotoxic T lymphocyte (CTL) response. “Inducing” an antigen-specific antibody response means increasing titer of the antigen-specific antibodies above a threshold level such as a pre-administration baseline titer or a seroprotective level. “Inducing” an antigen-specific CTL response means increasing frequency of antigen-specific CTL found in peripheral blood above a pre-administration baseline frequency.

Analysis (both qualitative and quantitative) of the immune response can be by any method known in the art, including, but not limited to, measuring antigen-specific antibody production (including measuring specific antibody subclasses), activation of specific populations of lymphocytes such as B cells and helper T cells, production of cytokines such as IFN-alpha, IFN-gamma, IL-6, IL-12 and/or release of histamine. Methods for measuring antigen-specific antibody responses include enzyme-linked immunosorbent assay (ELISA). Activation of specific populations of lymphocytes can be measured by proliferation assays, and with fluorescence-activated cell sorting (FACS). Production of cytokines can also be measured by ELISA. In some embodiments, methods of stimulating an immune response comprise stimulation of interleukin-1beta (IL-1β) secretion, interferon-gamma (IFN-γ) secretion, and/or tumor necrosis factor-alpha (TNF-α) secretion by monocyte-derived dendritic cells or peripheral blood mononuclear cells. In some preferred embodiments, at least 50%, 55%, 60%, 65%, 70% or 75% of the cells contacted with a composition of the present disclosure remain viable at 40-56 hours (or about 48 hours) post-contact.

In some embodiments, the methods are suitable for stimulating an anti-tumor immune response. In other embodiments, the methods are suitable for stimulating an anti-microbe immune response. In some embodiments, the anti-microbe response is an anti-bacterial immune response. In some embodiments, the anti-microbe response is an anti-fungal immune response. In some embodiments, the anti-microbe response is, an anti-viral immune response. In some embodiments, the anti-microbe response is an anti-protozoan immune response.

### B. Treating or Preventing Disease

The present disclosure further provides methods of treating or preventing a disease in an individual, comprising administering to the individual a composition or formulation described herein in an amount sufficient to treat or prevent a disease in the individual. In some embodiments, the disease is cancer. In some embodiments, the disease is abnormal cell proliferation. In other embodiments, the disease is an infectious disease.

In one aspect, the methods may comprise administering a composition comprising an LPC compound and at least one further lipid to a subject in need thereof, wherein the LPC and the at least one further lipid are part of a lipid nanoparticle (LNP). In another aspect, the methods involve adoptive cell therapy, and comprise administering a composition comprising a dendritic cell, such as a hyperactivated dendritic cell, an LPC compound, and a further lipid to a subject in need thereof, wherein the LPC and the at least one further lipid are part of a lipid nanoparticle (LNP). In some embodiments, the compositions further comprise a PRR agonist, an antigen, an adjuvant, or any combination thereof.

In some embodiments, the methods involve treating cancer in an individual or otherwise treating a mammalian subject with cancer. In some embodiments, the methods comprise: a) preparing an immunogenic composition comprising a tumor cell lysate, an isolated lysophosphatidylcholine (LPC) having a single acyl chain, at least one further lipid, and a toll-like receptor 7/8 (TLR7/8) agonist, wherein the tumor cell lysate is or has been prepared from a sample of a tumor obtained from the subject with cancer, the acyl chain is a C13-C22 acyl chain or a C13-C24 acyl chain, and the LPC and the at least one further lipid are part of a lipid nanoparticle (LNP); and b) administering to the subject an effective amount of the immunogenic composition. In some embodiments, the cancer is a hematologic cancer, such as a lymphoma, a leukemia, or a myeloma. In other embodiments, the cancer is a non-hematologic cancer, such as a sarcoma, a carcinoma, or a melanoma. In some embodiments, the cancer is malignant.

In some embodiments, the methods involve inhibiting abnormal cell proliferation in an individual. “Abnormal cell proliferation” refers to proliferation of a benign tumor or a malignant tumor. The malignant tumor may be a metastatic tumor.

In some embodiments, the methods involve treating or preventing an infectious disease in an individual. In some embodiments, the infectious disease is caused by a viral infection. In other embodiments, the infectious disease is caused by a bacterial infection. In further embodiments, the infectious disease is caused by a fungal infection. In still further embodiments, the infectious disease is caused by a protozoal infection. Of particular importance are infectious diseases caused by zoonotic pathogens that infect humans as well as other animals such as mammals or birds. In some embodiments, the zoonotic pathogen is transmitted to humans via an intermediate species (vector).

### ENUMERATED EMBODIMENTS

1. A composition comprising an isolated lysophosphatidylcholine (LPC) with a single acyl chain, at least one further lipid, and a TLR7/8 agonist, wherein
the acyl chain is a C13-C22 acyl chain or a C13-C24 acyl chain,
the at least one further lipid is selected from the group consisting of an ionizable lipid, a cationic lipid, a further phospholipid, a pegylated lipid, a structural lipid, and mixtures thereof, and
the LPC and the at least one further lipid are part of a lipid nanoparticle (LNP).

2. The composition of embodiment 1, wherein the acyl chain is a C18-C22 acyl chain or a C21-C24 acyl chain.

3 The composition of embodiment 1 or embodiment 2, further comprising an antigen.

4. The composition of any one of embodiments 1-3, further comprising dendritic cells.

5. A composition comprising an isolated lysophosphatidylcholine (LPC) with a single acyl chain, at least one further lipid, and an antigen, wherein
the acyl chain is a C21-C24 acyl chain,
the at least one further lipid is selected from the group consisting of an ionizable lipid, a cationic lipid, a further phospholipid, a pegylated lipid, a structural lipid, and mixtures thereof, and
the LPC and the at least one further lipid are part of a lipid nanoparticle (LNP).

6. The composition of embodiment 5, further comprising dendritic cells.

7. The composition of embodiment 5 or embodiment 6, further comprising a TLR7/8 agonist.

8. A composition comprising an isolated lysophosphatidylcholine (LPC) with a single acyl chain, at least one further lipid, and dendritic cells, wherein
the acyl chain is a C21-C24 acyl chain,
the at least one further lipid is selected from the group consisting of an ionizable lipid, a cationic lipid, a further phospholipid, a pegylated lipid, a structural lipid, and mixtures thereof, and
the LPC and the at least one further lipid are part of a lipid nanoparticle (LNP).

9. The composition of embodiment 8, further comprising a TLR7/8 agonist.

10. The composition of embodiment 8 or embodiment 9, further comprising an antigen.

11. The composition of any one of embodiments 1-10, wherein the acyl chain is a C22 acyl chain.

12. The composition of any one of embodiments 1-11, wherein the acyl chain is fully saturated.

13. The composition of any one of embodiments 1-12, wherein the LPC comprises 1-behenoyl-2-hydroxy-sn-glycero-3-phosphocholine [LPC(22:0)].

14. The composition of any one of embodiments 1-13, wherein the TLR7/8 agonist is a small molecule with a molecule weight of 900 daltons or less.

15. The composition of embodiment 14, wherein the TLR7/8 agonist comprises an imidazoquinoline compound.

16. The composition of embodiment 15, wherein the TLR7/8 agonist comprises resiquimod (R848).

17. The composition of embodiment 14 or embodiment 15, wherein the TLR7/8 agonist does not inhibit NLR family pyrin domain containing 3 (NLRP3).

18. The composition of embodiment 13, wherein the LPC comprises LPC(22:0), and the TLR7/8 agonist comprises resiquimod (R848).

19. The composition of any one of embodiments 1-18, wherein the antigen is present in a biological sample obtained from an individual.

20. The composition of embodiment 19, wherein the biological sample comprises biopsy tissue.

21. The composition of embodiment 19, wherein the biological sample comprises cells.

22. The composition of embodiment 19, wherein the biological sample does not comprise cells.

23. The composition of embodiment 19, wherein the biological sample comprises pus from an abscess.

24. The composition of any one of embodiments 1-23, wherein the antigen comprises a proteinaceous antigen.

25. The composition of embodiment 24, wherein the antigen comprises a tumor antigen.

26. The composition of embodiment 25, wherein the tumor antigen comprises a synthetic or recombinant neoantigen.

27. The composition of embodiment 26, wherein the tumor antigen comprises a tumor cell lysate.

28. The composition of embodiment 24, wherein the antigen comprises a microbial antigen and the microbial antigen comprises one or more of a viral antigen, a bacterial antigen, a protozoan antigen, and a fungal antigen.

29. The composition of embodiment 28, wherein the microbial antigen comprises a purified or recombinant surface protein.

30. The composition of embodiment 28, wherein the microbial antigen comprises an inactivated, whole virus.

31. The composition of any one of embodiments 1-30, wherein the composition comprises liposomes.

32. The composition of any one of embodiments 1-31, wherein the composition does not comprise lipopolysaccharide (LPS) or monophosphoryl lipid A (MPLA).

33. The composition of any one of embodiments 1-32, wherein the composition does not comprise oxidized 1-palmitoyl-2-arachidonoyl-sn-glycero-3-phosphorylcholine (oxPAPC) or a species of oxPAPC.

34. The composition of embodiment 33, wherein the composition does not comprise 2-[[(2R)-2-[(E)-7-carboxy-5-hydroxyhept-6-enoyl]oxy-3-hexadecanoyloxypropoxy]-hydroxyphosphoryl]oxyethyl-trimethylazanium (HOdiA-PC), [(2R)-2-[(E)-7-carboxy-5-oxohept-6-enoyl]oxy-3-hexadecanoyloxypropyl] 2-(trimethylazaniumyl) ethyl phosphate (KOdiA-PC), 1-palmitoyl-2-(5-hydroxy-8-oxo-octenoyl)-sn-glycero-3-phosphorylcholine (HOOA-PC), 2-[(2R)-2-[(E)-5,8-dioxooct-6-enoyl]oxy-3-hexadecanoyloxypropoxy]-hydroxyphosphoryl]oxyethyl-trimethylazanium (KOOA-PC), [(2R)-3-hexadecanoyloxy-2-(5-oxopentanoyloxy) propyl] 2-(trimethylazaniumyl) ethyl phosphate (POVPC), [(2R)-2-(4-carboxybutanoyloxy)-3-hexadecanoyloxy propyl] 2-(trimethylazaniumyl) ethyl phosphate (PGPC), [(2R)-3-hexadecanoyloxy-2-[4-[3-[(E)-[2-[(Z)-oct-2-enyl]-5-oxocyclopent-3-en-1-ylidene]methyl]oxiran-2-yl]butanoyloxy]propyl] 2-(trimethylazaniumyl) ethyl phosphate (PECPC), [(2R)-3-hexadecanoyloxy-2-[4-[3-[(E)-[3-hydroxy-2-[(Z)-oct-2-enyl]-5-oxocyclopentylidene]methyl]oxiran-2-yl]butanoyloxy]propyl] 2-(trimethylazaniumyl) ethyl phosphate (PEIPC) and/or 1-palmitoyl-2-azelaoyl-sn-glycero-3-phosphocholine (PAzePC).

35. The composition of any one of embodiments 1-34, further comprising an adjuvant, wherein the adjuvant comprises an aluminum salt adjuvant, a squalene-in-water emulsion, a saponin, or combinations thereof.

36. A pharmaceutical formulation comprising the composition of any one of embodiments 1-35 and a pharmaceutically acceptable excipient.

37. A method for production of hyperactivated dendritic cells, the method comprising contacting the dendritic cells with an effective amount of a composition comprising an isolated lysophosphatidylcholine (LPC) with a single C13-C22 acyl chain or a C13-C24 acyl chain, at least one further lipid, and a TLR7/8 agonist, for production of hyperactivated dendritic cells, wherein the hyperactivated dendritic cells secrete IL-1beta without undergoing pyroptosis, the at least one further lipid is selected from the group consisting of an ionizable lipid, a cationic lipid, a further phospholipid, a pegylated lipid, a structural lipid, and mixtures thereof, and the LPC and the at least one further lipid are part of a lipid nanoparticle (LNP).

38. The method of embodiment 37, wherein the dendritic cells are contacted ex vivo with the composition of any one of embodiments 1-35 or the formulation of embodiment 36.

39. The method of embodiment 37, wherein the dendritic cells are contacted in vivo with the formulation of embodiment 36.

40. A pharmaceutical formulation comprising at least 10³, 10⁴, 10⁵ or 10⁶ of the hyperactivated dendritic cells produced by the method of embodiment 38, and a pharmaceutically acceptable excipient.

41. A method of stimulating an immune response against an antigen, comprising administering an effective amount of the formulation of embodiment 36 to an individual in need thereof to stimulate the immune response against the antigen.

42. A method of treating cancer, comprising administering an effective amount of the formulation of embodiment 36 to an individual in need thereof to treat the cancer.

43. A method of inhibiting abnormal cell proliferation, comprising administering an effective amount of the formulation of embodiment 36 to an individual in need thereof to inhibit abnormal cell proliferation.

44. A method of treating an infectious disease, comprising administering an effective amount of the formulation of embodiment 36 to an individual in need thereof to treat the infectious disease.

45. Use of the formulation of embodiment 36 for inducing an immune response against the antigen in an individual in need thereof.

46. Use of the formulation of embodiment 36 for inducing an anti-tumor immune response in an individual in need thereof, wherein the individual is or was tumor-bearing.

47. Use of the formulation of embodiment 36 for inducing an anti-microbe immune response in an individual in need thereof, wherein the individual is infected with the microbe or has not been exposed to the microbe.

48. The composition, formulation, method or use of any one of embodiments 19-47, wherein the individual is a mammalian subject.

49. The composition, formulation, method or use of any one of embodiments 19-47, wherein the individual is a human subject.

50. A method of preparing an immunogenic composition, the method comprising:
a) obtaining a tumor cell-enriched suspension from a tumor;
b) lysing cells from the tumor cell-enriched suspension to obtain a tumor cell lysate; and
c) contacting the tumor cell lysate with a composition comprising an isolated lysophosphatidylcholine (LPC) having a single acyl chain, at least one further lipid, and a toll-like receptor 7/8 (TLR7/8) agonist, to obtain the immunogenic composition, wherein
the acyl chain is a C13-C22 acyl chain or a C13-C24 acyl chain,
the at least one further lipid is selected from the group consisting of an ionizable lipid, a cationic lipid, a further phospholipid, a pegylated lipid, a structural lipid, and mixtures thereof, and
the LPC and the at least one further lipid are part of a lipid nanoparticle (LNP).

51. The method of embodiment 50, wherein step a) comprising depleting leukocytes from the tumor cell-enriched suspension, optionally wherein the leukocytes are depleted by negative selection using an anti-CD45 antibody.

52. The method of embodiment 50 or embodiment 51, wherein the cells are lysed in step b) by one or more freeze-thaw cycles.

53. The method of any one of embodiments 50-52, wherein the acyl chain is a fully saturated C18-C22 acyl chain or a fully saturated C18-C24 acyl chain.

54. The method of embodiment 53, wherein the LPC comprises 1-behenoyl-2-hydroxy-sn-glycero-3-phosphocholine [LPC(22:0)].

55. The method of any one of embodiments 50-54, wherein the TLR7/8 agonist is a small molecule with a molecule weight of 900 daltons or less.

56. The method of embodiment 55, wherein the TLR7/8 agonist comprises an imidazoquinoline compound.

57. The method of embodiments 56, wherein the TLR7/8 agonist comprises resiquimod (R848).

58. The method of embodiment 55 or embodiment 56, wherein the TLR7/8 agonist does not inhibit NLR family pyrin domain containing 3 (NLRP3).

59. The method of embodiment 54, wherein the LPC comprises LPC(22:0), and the TLR7/8 agonist comprises resiquimod (R848).

60. The method of any one of embodiments 50-59, further comprising before step a) obtaining a sample from the tumor from a mammalian subject with cancer and preparing the suspension of cells from the sample.

61. An immunogenic composition prepared by the method of any one of embodiments 50-60.

62. A method of eliciting an anti-cancer immune response, the method comprising: administering to a mammalian subject with cancer an effective amount of the immunogenic composition of embodiment 61.

63. The method of embodiment 62, wherein the anti-cancer immune response comprises cellular immune response.

64. The method of embodiment 63, wherein the anti-cancer immune response comprises cancer antigen-induced IL-1beta secretion and/or activation of CD8+T lymphocytes.

65. The method of any one of embodiments 62-64, wherein the cancer is a non-hematologic cancer.

66. The method of embodiment 65, wherein the non-hematologic cancer is a carcinoma, a sarcoma, or a melanoma.

67. The method of any one of embodiments 62-64, wherein the cancer is a lymphoma.

68. A method of treating cancer, the method comprising:
a) preparing an immunogenic composition comprising a tumor cell lysate, an isolated lysophosphatidylcholine (LPC) having a single acyl chain, at least one further lipid, and a toll-like receptor 7/8 (TLR7/8) agonist, wherein
the tumor cell lysate is or has been prepared from a sample of a tumor obtained from the mammalian subject with cancer,
the acyl chain is a C13-C22 acyl chain or a C13-C24 acyl chain,
the at least one further lipid is selected from the group consisting of an ionizable lipid, a cationic lipid, a further phospholipid, a pegylated lipid, a structural lipid, and mixtures thereof, and
the LPC and the at least one further lipid are part of a lipid nanoparticle (LNP); and
b) administering to the subject an effective amount of the immunogenic composition.

69. The method of any one of embodiments 62-68, wherein the acyl chain is a fully saturated C18-C22 acyl chain or a fully saturated C18-C24 acyl chain.

70. The method of embodiment 68, wherein the LPC comprises 1-behenoyl-2-hydroxy-sn-glycero-3-phosphocholine [LPC(22:0)].

71. The method of any one of embodiments 62-70, wherein the TLR7/8 agonist is a small molecule with a molecule weight of 900 daltons or less.

72. The method of embodiment 71, wherein the TLR7/8 agonist comprises an imidazoquinoline compound.

73. The method of embodiment 72, wherein the TLR7/8 agonist comprises resiquimod (R848).

74. The method of embodiment 70, wherein the LPC comprises 22:0 LPC, and the TLR7/8 agonist comprises resiquimod (R848).

75. The method of any one of claims 68-74, further comprising administering to the subject an effective amount of an additional therapeutic agent.

76. The method of embodiment 75, wherein the additional therapeutic agent comprises one or more of the group consisting of an immune checkpoint inhibitor, an antineoplastic agent, and radiation therapy.

77. A composition comprising an isolated lysophosphatidylcholine (LPC) with a single acyl chain, at least one further lipid, and a pathogen recognition receptor (PRR) agonist, wherein
the acyl chain is a C13-C22 acyl chain or a C13-C24 acyl chain,
the at least one further lipid is selected from the group consisting of an ionizable lipid, a cationic lipid, a further phospholipid, a pegylated lipid, a structural lipid, and mixtures thereof, and
the LPC and the at least one further lipid are part of a lipid nanoparticle (LNP).

78. The composition of embodiment 77, wherein the PRR agonist is an agonist of a toll-like receptor (TLR), a NOD-like receptor (NLR), a RIG-I-like receptor (RLR), or a C-type lectin receptor (CLR).

79. The composition of embodiment 77, Wherein the PRR agonist is an agonist of a cytosolic DNA sensor (CDS) or a stimulator of IFN genes (STING).

80. The composition of embodiment 77, wherein the PRR agonist comprises one or more of R848, TL8-506, LPS, Pam2CSK4, and ODN 2336.

81. The composition of any one of embodiments 77-80, further comprising an antigen.

82. The composition of any one of embodiments 77-81, further comprising dendritic cells.

83. A pharmaceutical formulation comprising the composition of any one of embodiments 77-82 and a pharmaceutically acceptable excipient.

84. A pharmaceutical formulation comprising an isolated lysophosphatidylcholine (LPC) with a single acyl chain, at least one further lipid, and a pharmaceutically acceptable excipient, wherein
the acyl chain is a C21-C24 acyl chain,
the at least one further lipid is selected from the group consisting of an ionizable lipid, a cationic lipid, a further phospholipid, a pegylated lipid, a structural lipid, and mixtures thereof, and
the LPC and the at least one further lipid are part of a lipid nanoparticle (LNP).

85. The pharmaceutical formulation of embodiment 83 or embodiment 84, wherein the acyl chain is a fully saturated C22 acyl chain.

86. The pharmaceutical formulation of embodiment 85, wherein the LPC comprises 1-behenoyl-2-hydroxy-sn-glycero-3-phosphocholine [LPC(22:0)].

87. A composition for hyperactivation of human dendritic cells, comprising an isolated lysophosphatidylcholine (LPC) with a single acyl chain, at least one further lipid, and a pathogen recognition receptor (PRR) agonist, wherein
the acyl chain is C22 acyl chain,
the at least one further lipid is selected from the group consisting of an ionizable lipid, a cationic lipid, a further phospholipid, a pegylated lipid, a structural lipid, and mixtures thereof,
the LPC and the at least one further lipid are part of a lipid nanoparticle (LNP), and
the composition is effective for achieving a higher level of dendritic cell hyperactivation than a comparator composition comprising PGPC in place of the LPC.

88. The composition of embodiment 87, wherein the higher level of dendritic cell hyperactivation comprises induction of IL-1beta secretion from the human dendritic cells in vitro at a level that is at least 2, 3 or 4 fold higher when contacted with the composition comprising the LPC and the PRR agonist than when contacted with the comparator composition comprising the PGPC and the PRR agonist, wherein the PRR agonist is LPS.

89. The composition of embodiment 88, wherein the concentration of the LPC and the concentration of the PGPC are the same concentration in a range of from about 10 μM to about 80 μM, and the LPS is present at a concentration of 1 μg/ml in both the composition and the comparator composition.

90. The composition of embodiment 88, wherein the higher level of dendritic cell hyperactivation comprises a lipid activity index for IL-1beta secretion from the human dendritic cells for the composition comprising the LPC and the PRR agonist that is at least 4, 5 or 6 fold higher in activity units than that of the comparator composition comprising the PGPC and the PRR agonist.

91. The composition, formulation, method or use of any one of embodiments 19-47, wherein the individual is a canine subject.

92. The composition, formulation, method or use of any one of embodiments 60-90, wherein the mammalian subject is a human patient.

93. The composition, formulation, method or use of any one of embodiments 60-90, wherein the mammalian subject is a non-human patient.

94. The composition, formulation, method or use of any one of embodiments 60-90, wherein the mammalian subject is a canine patient.

95. The composition, formulation, method or use of any one of embodiment 1-90 or 92, wherein the dendritic cells are human dendritic cells.

96. The composition, formulation, method or use of any one of embodiment 1-91 or 94, wherein the dendritic cells are canine dendritic cells.

97. The composition, formulation, method or use of embodiment 95 or embodiment 96, wherein the dendritic cells are present in a composition comprising peripheral blood mononuclear cells (PBMCs).

98. The composition, formulation, method or use of any one of embodiments 37-49 or embodiment 91, wherein the hyperactivated dendritic cells secrete one or both of IFNγ and TNFα.

99. The composition, formulation, method or use of any one of embodiments 1-98, wherein the at least one further lipid comprises one or both of a further phospholipid and a structural lipid, optionally wherein the further phospholipid comprises 1,2-distearoyl-sn-glycero-3-phosphocholine (DSPC), and the structural lipid comprises cholesterol.

100. The composition, formulation, method or use of embodiment 99, wherein the at least one further lipid comprises a pegylated lipid, optionally wherein the pegylated lipid comprises polyethylene glycol [PEG] 2000 dimyristoyl glycerol [DMG].

101. The composition, formulation, method or use of embodiment 99 or embodiment 100, wherein the at least one further lipid comprises an ionizable lipid, optionally wherein the ionizable lipid comprises 4-(dimethylamino)-butanoic acid, (10Z,13Z)-1-(9Z,12Z)-9,12-octadecadien-1-yl-10,13-nonadecadien-1-yl ester (DLin-MC3-DMA) or analogs or derivatives thereof.

102. A composition comprising a lipid nanoparticle (LNP), wherein the LNP comprises a first phospholipid, and at least one lipid selected from the group consisting of an ionizable lipid, a second phospholipid, a pegylated lipid, a structural lipid, and mixtures thereof, wherein the first phospholipid comprises a lysophosphatidylcholine (LPC) with a single acyl chain, and the acyl chain is a C13-C24 acyl chain.

103. A composition comprising a lipid nanoparticle (LNP), wherein the LNP comprises a first phospholipid, an ionizable lipid, a second phospholipid, a pegylated lipid, and a structural lipid, wherein the first phospholipid comprises a lysophosphatidylcholine (LPC) with a single acyl chain, and the acyl chain is a C13-C24 acyl chain.

104. The composition of any one of embodiments 1-103, wherein the ionizable lipid comprises:
i) 8-[(2-hydroxyethyl) [6-oxo-6-(undecyloxy) hexyl]amino]-octanoic acid, 1-octylnonyl ester (SM-102) or analogs or derivatives thereof; and/or 6-((2-hexyldecanoyl)oxy)-N-(6-((2-hexyldecanoyl)oxy) hexyl)-N-(4-hydroxybutyl) hexan-1-aminium (ALC-0315) or analogs or derivatives thereof; or
ii) (6Z,9Z,28Z,31Z)-heptatriaconta-6,9,28,31-tetraen-19-yl 4-(dimethylamino) butanoate (DLin-MC3-DMA) or analogs or derivatives thereof.

105. The composition of any one of embodiments 1-104, wherein the pegylated lipid is selected from the group consisting of a PEG-modified phosphatidyiethanolamine, a PEG-modified phosphatide acid, a PEG-modified ceramide, a PEG-modified dialkylamine, a PEG-modified diacylglycerol, a PEG-modified dialkylglyerol, and combinations thereof.

106. The composition of any one of embodiments 1-104, wherein the pegylated lipid comprises polyethylene glycol [PEG] 2000 dimyristoyl glycerol [DMG].

107. The composition of any one of embodiments 1-106, wherein the structural lipid is selected from the group consisting of cholesterol, fecosterol, sitosterol, ergosterol, campesterol, stigmasterol, brassicasterol, tomatidine, ursolic acid, alpha-tocopherol, and combinations thereof.

108. The composition of any one of embodiments 1-106, wherein the structural lipid comprises cholesterol.

109. The composition of any one of embodiments 1-108, wherein the further phosholipid or the second phospholipid comprises a hydrophilic head moiety selected from the group consisting of phosphatidyl choline, phosphatidyl ethanolamine, phosphatidyl glycerol, phosphatidyl serine, phosphatidic acid, 2-lysophosphatidyl choline, and sphingomyelin.

110. The composition of embodiment 1-108, wherein the further phosholipid or the second phospholipid comprises one or more fatty acid tail moieties selected from the group consisting of lauric acid, myristic acid, myristoleic acid, palmitic acid, palmitoleic acid, stearic acid, oleic acid, linoleic acid, alpha-linolenic acid, erucic acid, arachidic acid, arachidonic acid, phytanoic acid, eicosapentaenoic acid, behenic acid, docosapentaenoic acid, and docosahexaenoic acid.

111. The composition of any one of embodiments 1-108, wherein the further phosholipid or the second phospholipid is selected from the group consisting of
1,2-dilinoleoyl-sn-glycero-3-phosphocholine (DLPC),
1,2-dimyristoyl-sn-glycero-phosphocholine (DMPC),
1,2-dioleoyl-sn-glycero-3-phosphocholine (DOPC),
1,2-dipalmitoyl-sn-glycero-3-phosphocholine (DPPC),
1,2-distearoyl-sn-glycero-3-phosphocholine (DSPC),
1,2-diundecanoyl-sn-glycero-phosphocholine (DUPC),
1-palmitoyl-2-oleoyl-sn-glycero-3-phosphocholine (POPC),
1,2-di-O-octadecenyl-sn-glycero-3-phosphocholine,
1-oleoyl-2-cholesterylhemisuccinoyl-sn-glycero-3-phosphocholine,
1,2-dilinolenoyl-sn-glycero-3-phosphocholine,
1,2-diarachidonoyl-sn-glycero-3-phosphocholine,
1,2-didocosahexaenoyl-sn-glycero-3-phosphocholine,
1,2-dioleoyl-sn-glycero-3-phosphoethanola mine (DOPE),
1,2-diphytanoyl-sn-glycero-3-phosphoethanolamine,
1,2-distearoyl-sn-glycero-3-phosphoethanolamine,
1,2-dilinoleoyl-sn-glycero-3-phosphoethanolamine,
1,2-dilinolenoyl-sn-glycero-3-phosphoethanolamine,
1,2-diarachidonoyl-sn-glycero-3-phosphoethanolamine,
1,2-didocosahexaenoyl-sn-glycero-3-phosphoethanolamine,
1,2-dioleoyl-sn-glycero-3-phospho-rac-(1-glycerol) sodium salt (DOPG),
sphingomyelin, and
combinations thereof.

112. The composition of any one of embodiments 1-108, wherein the further phosholipid or the second phospholipid comprises 1,2-distearoyl-sn-glycero-3-phosphocholine (DSPC).

113. The composition of any one of embodiments 1-112, wherein the at least one further lipid comprises: i) a cationic lipid, and comprises or further comprises ii) a neutral or anionic lipid.

114. The composition of embodiment 113, wherein the cationic lipid comprises one or both of:
i) 1,2-di-O-octadecenyl-3-trimethylammonium propane (DOTMA) or analogs or derivatives thereof; and
ii) 1,2-dioleoyl-3-trimethylammonium propane (DOTAP) or analogs or derivatives thereof.

115. The composition of embodiment 113 or embodiment 114, wherein the neutral or anionic lipid comprises:
i) 1,2-di-(9Z-octadecenoyl)-sn-glycero-3-phosphoethanolamine (DOPE) or analogs or derivatives thereof; and/or
ii) cholesterol or analogs or derivatives thereof; and/or
iii) 1,2-dioleoyl-sn-glycero-3-phosphocholine (DOPC) or analogs or derivatives thereof.

116. The composition of any one of embodiments 102-115, wherein the acyl chain of the LPC is a C21-C24 acyl chain.

117. The composition of any one of embodiments 102-115, wherein the acyl chain of the LPC is a C22 acyl chain.

118. The composition of any one of embodiments 102-117, wherein the composition further comprises a TLR7/8 agonist.

119. The composition of embodiment 118, wherein the TLR7/8 agonist comprises an imidazoquinoline compound.

120. The composition of embodiment 119, wherein the TLR7/8 agonist comprises resiquimod (R848).

121. The composition of embodiment 119, wherein the LPC comprises LPC(22:0), and the TLR7/8 agonist comprises resiquimod (R848) 122. The composition of any one of embodiments 102-121, wherein the composition further comprises an antigen.

123. The composition of embodiment 122, wherein the antigen is a tumor antigen or a neoantigen.

124. The composition of embodiment 122, wherein the antigen is a microbioal antigen, optionally wherein the microbial antigen a viral antigen, a bacterial antigen, a protozoan antigen, or a fungal antigen.

125. The composition, formulation, method or use of any one of embodiments 1-124, wherein the composition does not comprise isolated mRNA.

126. The composition, formulation, method or use of any one of embodiments 1-125, wherein the LNP has an effective diameter of less than about 500 nanometers, optionally from about 5 to about 500 nanometers, optionally from about 10 to about 400 nanometers, optionally from about 20 to about 300 nanometers, or optionally from about 25 to about 250 nanometers.

127. The composition, formulation, method or use of embodiment 126, wherein the LNP has an effective diameter of less than about 250 nanometers.

128. The composition, formulation, method or use of embodiment 127, wherein the LNP has an effective diameter of less than about 125 nanometers.

129. The composition, formulation, method or use of embodiment 128, wherein the LNP has an effective diameter of from about 10 to about 110 nanometers 130. The composition, formulation, method or use of any one of embodiments 1-129, wherein the composition does not comprise a surfactant.

### EXAMPLES

Abbreviations: CDS (cytosolic DNA sensor); CLR (C-type lectin receptor); DAMP (damage-associated molecular pattern); DC (dendritic cell); dLN (draining lymph node); DLS (dynamic light scattering); DMG-PEG-2000 (polyethylene glycol [PEG] 2000 dimyristoyl glycerol [DMG]; DSPC (1,2-distearoyl-sn-glycero-3-phosphocholine); ELSD (evaporative light scattering detector); FLT3L (Fms-related tyrosine kinase 3 ligand); HOdiA-PC (1-palmitoyl-2-(5-hydroxy-8-oxo-6-octenedioyl)-sn-glycero-3-phosphatidylcholine); HOOA-PC (1-palmitoyl-2-(5-hydroxy-8-oxooct-6-enoyl)-sn-glycero-3-phosphocholine); IFNγ (interferon-gamma); IL-1b/IL1-beta/IL-1β (Interleukin-1beta); KOdiA-PC (1-(palmitoyl)-2-(5-keto-6-octene-dioyl) phosphatidylcholine); KOOA-PC (1-palmitoyl-(5-keto-8-oxo-6-octenoyl)-sn-glycero-3-phosphocholine); LNP (lipid nanoparticle); LPC/Lyso PC (lysophosphatidylcholine); Lyso PC (22:0) (1-behenoyl-2-hydroxy-sn-glycero-3-phosphocholine); LPS (lipopolysaccharide); MC3 ((6Z,9Z,28Z,31Z)-heptatriaconta-6,9,28,31-tetraen-19-yl 4-(dimethylamino) butanoate, also referred to as DLin-MC3-DMA); moDC (monocyte derived dendritic cell); MPLA (monophosphoryl lipid A); NLR (NOD-like receptor); oxPAPC (oxidized 1-palmitoyl-2-arachidonyl-sn-glycero-3-phosphorylcholine); PAMP (pathogen-associated molecular pattern); PBMCs (peripheral blood mononuclear cells); PGPC (1-palmitoyl-2-glutaryl-sn-glycero-3-phosphocholine); POVPC (1-palmitoyl-2-(5′-oxo-valeroyl)-sn-glycero-3-phosphocholine); PRR (pathogen recognition receptor); RLR (RIG-I-like receptor); R848 (resiquimod); SC (subcutaneously); STING (stimulator of IFN genes); TNFα (tumor necrosis factor-alpha); and TLR (toll-like receptor).

Although the present disclosure has been described in some detail by way of illustration and example for purposes of clarity and understanding, it will be apparent to those skilled in the art that certain changes and modifications may be practiced. Therefore, the following examples should not be construed as limiting the scope of the present disclosure, which is delineated by the appended claims.

### Example 1: Combination of a Lysophosphatidylcholine (LPC) with a Single Acyl Chain and a TLR7/8 agonist Hyperactivates Mammalian Peripheral Blood Mononuclear Cells

This example describes the hyperactivation of canine and human peripheral blood mononuclear cells (PBMCs) with a lipid DAMP in combination with a small molecule PAMP.

### Materials and Methods

Isolation of PBMCs from Whole Blood. PBMCs were isolated from whole blood using density gradient centrifugation with Ficoll-Paque PLUS (Cytivia). Whole blood was diluted 1:1 with PBS, layered on top of Ficoll-Paque PLUS and centrifuged at 1000×g for 30 minutes at room temperature. PBMCs were collected, washed twice in PBS, and incubated with Ack lysis buffer (Lonza) to remove any remaining red blood cells.

Cell Culture and Stimulation. Immediately following isolation, PBMCs were plated in RPMI medium containing 10% FBS, 50 units/mL penicillin, 50 mg/mL streptomycin, 2 mM L-glutamine, 1 mM sodium pyruvate, and 50 mM beta-mercaptoethanol (R10 media). Cells were plated at 1×10⁵ (canine cells) or 1×10⁶ (human cells) per well in 96-well flat bottom tissue culture plates. Lyophilized Vaccigrade R848 (Invivogen) was reconstituted and diluted according to manufacturer's recommendations and added to cells at a final concentration of 1 μg/mL. Immediately following, 22:0 LYSO PC was added to cells at a final concentration of 82.5 μM. Additional innate agonists were diluted in R10 media according to manufacturer's recommendations and added to the cells as follows: human GM-CSF (Peprotech) was added at a final concentration of 10 ng/ml; 2′3′ cGAMP (Invivogen) was added at a final concentration of 15 μg/mL; LPS, serotype O55: B5 (Enzo Life Sciences) was added at a final concentration of 1 μg/mL; Alum hydroxide (Invivogen) was added at a final concentration of 30 μg/mL. Cells were incubated at 37° C., 5% CO2 for two days. Cell cultures were then used for endpoint analyses.

Endpoint Analyses. After culturing PBMCs with PAMPs and DAMPs for two days, supernatant and cell samples were collected for analysis. Cells in culture were pelleted by centrifugation at 400×g for 5 minutes. Half of the media volume in the wells was collected for cytokine quantification by Enzyme-Linked Immunosorbent Assay (ELISA) or Lumit™ Bioluminescent assay, while the remaining media and cells were used to quantify cell viability by assessing metabolic activity.

Quantification of Cytokine Secretion. IL-1β secretion from human PBMCs was assessed using one of the following kits: ELISA MAX Deluxe Set Human IL-1β kit (Biolegend), Invitrogen Human IL-1β kit, or the Lumit™ Human IL-1β Immunoassay (Promega). IFNγ secretion from human PBMCs was assessed using the ELISA MAX Deluxe Set Human IFNγ (Biolegend) and TNFα secretion from human PBMCs was assessed using the Human TNFα Uncoated ELISA kit (Invitrogen). ELISAs were performed according to manufacturer's instructions with the following modifications: i) total sample+buffer volume for incubation was reduced from 100 μL to 50 μL; ii) the top standard was prepared at 500 μg/mL, with two-fold dilutions to 7.8 pg/mL; and iii) sample incubation was completed overnight at 4C on an orbital shaker. Lumit™ assays were performed according to manufacturer's instructions. IL-1β secretion from canine PBMCs was assessed using the Canine IL-1β/IL-1F2 DuoSet ELISA (R&D) according to manufacturer's instructions with the following modifications: i) total sample+buffer volume for incubation was reduced from 100 μL to 50 μL; ii) sample incubation was completed overnight at 4° C. on an orbital shaker. For all ELISAs, absorbance was measured at 450 nm, with a 570 nm correction, using a Spectramax M5e plate reader (Molecular Devices). For Lumit™ assays, luminescence was measured on all wavelengths using a Spectramax M5e plate reader (Molecular Devices) with an integration time of 500 ms. To determine cytokine concentrations in supernatants, sample concentrations were interpolated using a standard curve via 4PL analysis on GraphPad Prism 9 (GraphPad Software). The interpolated results of samples were then adjusted for any dilutions made to the supernatant.

Quantification of Cell Viability. Cell viability was assessed by quantifying the presence of ATP as an indicator of metabolically active cells using the CellTiter-Glo Luminescent Cell Viability Assay (Promega). Metabolic activity was assessed following manufacturer's instructions. The CellTiter-Glo reagent was mixed with the cell pellets and fresh media then transferred to a white, opaque 96-well plate. Luminescence was measured on all wavelengths on a Spectramax M5e plate reader (Molecular Devices) using an integration time of 500 ms. Percent viability was calculated relative to the control condition of PBMCs treated with R848.

Statistical Analyses. For each condition, cells from each donor were plated for testing in triplicate. For cytokine quantification, triplicate values were used for interpolation and data was plotted as total concentration (pg/mL) or fold change per donor relative to the control condition of R848 alone. For viability quantification, each donor triplicate was averaged, and the average was used as one donor measurement. Multiple donors were tested and each data point on the column graphs represents the value for a donor. To test for differences in test conditions, test results were compared to the control condition of R848 alone. P-values were calculated using a mixed-effects one-way ANOVA, with corrections for multiple comparisons using a Dunnett's test.

Results—Treatment with 22:0 LYSO PC and R848 Hyperactivates Canine PBMCs

The combination of 22:0 LYSO PC (DAMP) and the TLR7/8 agonist R848 (PAMP) was previously found to have potent hyperstimulatory activity in human moDCs. In order to assess whether this hyperstimulatory activity translates to other clinically relevant species, the ability of 22:0 LYSO PC+R848 to hyperactivate PBMCs isolated from canine whole blood was assessed. For each data set, PBMCs from multiple donors were used in lieu of moDCs due to the lack of canine-specific reagents available to induce bone fide canine moDCs. In brief, PBMCs were isolated from whole blood using density gradient centrifugation and then cultured for two days with the hyperactivating stimuli of interest.

After two days in culture, hyperactivation was assessed by quantification of IL-1β in cell culture supernatants and measurement of cell viability. When treated with 22:0 LYSO PC and R848 together, canine PBMCs secreted comparable or higher levels of IL-1β compared to every other stimuli tested, both as concentration per mL, as well as fold change per donor relative to R848 alone (FIG. 1A-1B). Consistent with previous studies showing that monocytes, which make up 5-10% of PBMCs, can release IL-1β in response to activation with R848, canine PBMCs had elevated levels of IL-1β secretion with R848 alone compared to untreated cells. The pyroptotic combination of LPS+Alum elicited high levels of IL-1β as expected. Notably, while PGPC+R848 elicited similar levels of IL-1β compared to R848 alone, neither GM-CSF nor 2′3′cGAMP induced significant IL-1β secretion from canine PBMCs compared to untreated cells.

Although IL-1β can be detected one day after hyperactivation of canine PBMCs in cell culture supernatants, cell viability was evaluated two days post-hyperactivation to ensure enduring viability after IL-1β secretion. 22:0 LYSO+R848 did not significantly reduce relative cell viability (FIG. 1C). Interestingly, PGPC in combination with R848 proved to be somewhat toxic to canine PBMCs, although it was not observed to be toxic to human moDCs or human PBMCs. However, interpreting observations made from testing mixed cell populations is challenging, as the viability of a specific cell population of interest (in this case, monocytes) cannot be determined from results obtained with the mixture. Together these data demonstrate that 22:0 LYSO+R848 elicits high levels of IL-1β secretion from canine PBMCs, which is indicative of hyperactivation.

Results—Treatment with 22:0 LYSO PC and R848 Hyperactivates Human PBMCs

Hyperactivation experiments were also performed with PBMCs isolated from whole blood obtained from human donors. In brief, PBMCs were isolated from whole blood by density gradient centrifugation from multiple human donors and cultured for two days with the hyperactivating stimuli of interest.

Human PBMCs, like human moDCs and canine PBMCs, secreted IL-1β at levels higher or comparable to all other stimuli tested (FIG. 2A-2B). Similar to canine PBMCs, human PBMCs secreted IL-1β in response to R848 alone due to monocyte activation, and this was elevated by addition of 22:0 LYSO PC. The pyroptotic combination of LPS+Alum elicited high levels of IL-1β as expected. Consistent with observations in canine PBMCs, PGPC+R848 did not induce substantially higher levels of IL-1β than R848 alone. GM-CSF did not induce levels of IL-1β secretion from human PBMCs significantly above background levels produced by untreated cells.

Viability of human PBMCs was also assessed two days post-hyperactivation to ensure enduring viability of human PBMCs after IL-1β secretion. No significant decreases in human PBMC viability were observed after treatment with any of the stimuli (FIG. 2C). However, interpreting observations made from testing mixed cell populations is challenging, as the viability of a specific cell population of interest (in this case, monocytes) cannot be determined from results obtained with the mixture. Together these data demonstrate that both human and canine PBMCs are hyperactivated by 22:0 LYSO PC+R848. Interestingly, canine PBMCs are hyperactivated to a greater extent by 22:0 LYSO PC+R848 than by PGPC+R848.

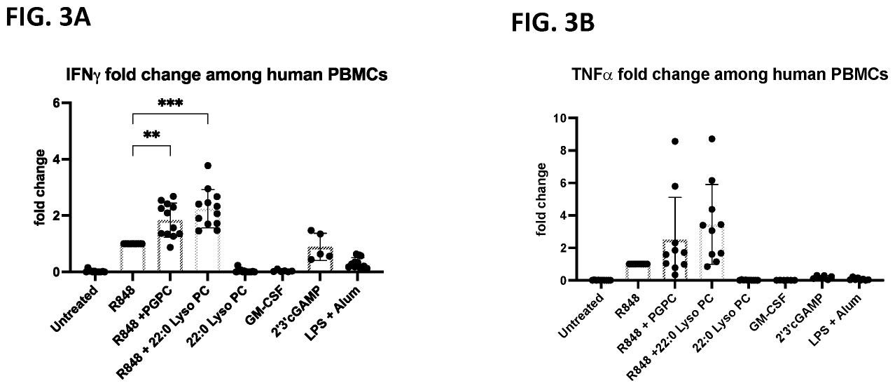

Because activated human PBMCs can secrete other cytokines in addition to IL-1β, the secretion of the pro-inflammatory cytokines IFNγ and TNFα in cell culture supernatants was measured two days post-hyperactivation. The combination of 22:0 LYSO PC+R848 induced the highest fold change per donor in both IFNγ secretion and TNFα secretion relative to R848 alone as compared to all other stimuli tested (FIG. 3A-3B). Notably, although LPS+Alum induced high levels of IL-1β secretion from human PBMCs (FIG. 3B), this combination of stimuli did not induce a fold increase in IFNγ or TNFα secretion. Moreover, neither GM-CSF nor 2′3′cGAMP elicited a substantial fold change in IFNγ secretion over R848 alone. These data indicate that the combination of 22:0 LYSO PC+R848 is superior at inducing secretion of the proinflammatory cytokines IFNγ and TNF from human PBMCs.

### Example 2: Preparation of Lipid Nanoparticles Comprising a Lysophosphatidylcholine (LPC)

This example describes preparation of lipid nanoparticles (LNPs) loaded with a hyperactivating lipid (e.g., 22:0 LYSO PC) in a microfluidic process.

### Materials and Methods

LNPs were synthesized using the NanoAssemblr® Ignite™ microfluidic instrument (Precision Nanosystems, Vancouver, BC, Canada). Initially, a kit containing Gen Voy-ILM™ ionizable lipid mix (Precision Nanosystems, Vancouver, BC, Canada) was used to produce LNPs. The kit without mRNA was used to build empty LNP vehicles, and hyperactivator loaded LNPs were generated by adding 22:0 Lyso PC to a molar ratio of 10% of the total LNP content. LNPs were also produced using individual components (without a kit) to determine if 22:0 Lyso PC loading into LNPs could be intentionally varied. LNPs were prepared by combining the following components with or without 1-behenoyl-2-hydroxy-sn-glycero-3-phosphocholine
(CAS Registry No. 125146-65-8, referred to herein as “22:0 Lyso PC”) (Avanti):
   (10Z,13Z)-1-(9Z,12Z)-9,12-octadecadien-1-yl-10,13-nonadecadien-1-yl ester
(CAS Registry No. 1224606-06-7, referred to herein as “DLin-MC3-DMA” or “MC3”) (Cayman Chemical);
   1,2-distearoyl-sn-glycero-3-phosphocholine
(CAS Registry No. 816-94-4, referred to herein as “DSPC”) (Avanti);
   cholesterol (Sigman); and
   1,2-dimyristoyl-rac-glycero-3-methoxypolyethylene glycol-2000
(CAS Registry No. 160743-62-4, referred to herein as “DMG-PEG2000”) (Avanti).
Lipids were first dissolved in ethanol and then combined following the molarity percentages shown in Table 2-1. Lipids in ethanol were combined with PBS, pH 7.4 at a 1:3 volumetric ratio. The NanoAssemblr® Ignite™ microfluidic instrument was programmed with a flow rate of 12 mL/min, a start waste of 0.35 mL, and an end waste of 0.05 mL. LNPs were washed in PBS, pH 7.4 to remove residual ethanol, and then concentrated using Amicon 10K MWCO centrifugal filters by spinning at 2000×g for 30 minutes.

TABLE 2-1
LNP Formulations\hat{ }

| | GenVoy | LPC | LNP | LPC | LNP | LPC | LPC |
|---|---|---|---|---|---|---|---|
| | Vehicle | GenVoy | Vehicle | LNP | Vehicle | LNP | LNP |
| Lipid | LNP | LNP | 1 | 1 | 2 | 2 | 3 |
| GenVoy-ILM | 100.0% | 90.0% | — | — | — | — | — |
| MC3 | — | — | 45.0% | 41.0% | — | — | — |
| DSPC | — | — | 15.0% | 10.0% | 50.0% | 25.0% | 10.0% |
| Cholesterol | — | — | 38.5% | 37.5% | 48.5% | 48.5% | 48.5% |
| DMG-PEG2000 | — | — | 1.5% | 1.5% | 1.5% | 1.5% | 1.5% |
| 22:0 Lyso PC | — | 10.0% | — | 10.0% | — | 25.0% | 40.0% |

\hat{ }Percent molarity of components of different LNP formulations. LNP 2 and LNP 3 formulations share the same vehicle (LNP Vehicle 2).

Loading of 22:0 Lyso PC into LNPs was assessed using HPLC. LNPs in PBS were frozen at −80° C., then lyophilized and stored at −20° C. until they could be quantified. LNPs were reconstituted in ethanol, and then mixed with water to dissolve the PBS. A seven point standard curve of 22:0 Lyso PC was prepared in ethanol with water and PBS added to match sample preparation. Standards and samples were filtered through a 0.45 μm filter prior to running on the HPLC. HPLC quantification was performed on using an Agilent 1260 Infinity II HPLC equipped with a 1260 Infinity II Evaporative Light Scattering Detector (ELSD). A Luna 5 μm NH2 100 Å, 150×4.6 mm LC Column (Phenomenex, Torrance, CA) with a column temperature of 30° C. was used to detect samples. Two eluents were used: A, 100% water; and B, 100% acetonitrile. An initial mobile phase composed of 5%/95% A/B was used to load the column, with a gradient reaching 24%/76% A/B after 2.5 min. A more shallow gradient was used from 2.5 to 6 minutes, with A/B slowly reaching 25%/75% during that time frame. A post time of 3 min was used to return the gradient to starting conditions prior to the next sample run. The flow rate was set to 1 mL/min, and the injection volume was 5 μL for samples and standards. The ELSD used an evaporator temperature of 80° C., a nebulizer temperature of 30° C., and a nitrogen gas flow rate of 0.9 standard liters per minute. Agilent CDS 2.6 software was used for HPLC instrument control, data acquisition, and processing.

The size of the LNPs was assessed using dynamic light scattering (DLS) on the NanoBrook Omni particle size and zeta potential analyzer (Brookhaven Instruments Corp., Holtsville, NY). Four measurements were made for each sample for 120 seconds each, with the first measurement made for each sample excluded from downstream analyses as time needed for sample equilibration. Data points on sizing graph represent individual replicate measurements from two preparations of LNPs.

### Results

Incorporation of 22:0 Lyso PC into LNPs was explored to test the effects of 22:0 Lyso PC on physical characteristics and biological activity of LNPs. Both loading level and the number of loaded LNPs were contemplated to be key variables affecting the 22:0 Lyso PC payload delivered to cells.

The Gen Voy-ILM™ ionizable lipid mix (Precision Nanosystems, Vancouver, BC, Canada) was used to produce LNPs with 10% 22:0 Lyso PC added (based on molar ratio) or without addition of 22:0 Lyso PC (empty vehicle LNPs). Additional LNPs were produced by combining the following components with or without 22:0 Lyso PC: MC3, DPSC, cholesterol, and DMG-PEG2000. All LNPs were produced by using the NanoAssemblr® Ignite™ microfluidic instrument (Precision Nanosystems, Vancouver, BC, Canada). The LNPs were subsequently purified using spin filtration to remove ethanol and unincorporated material.

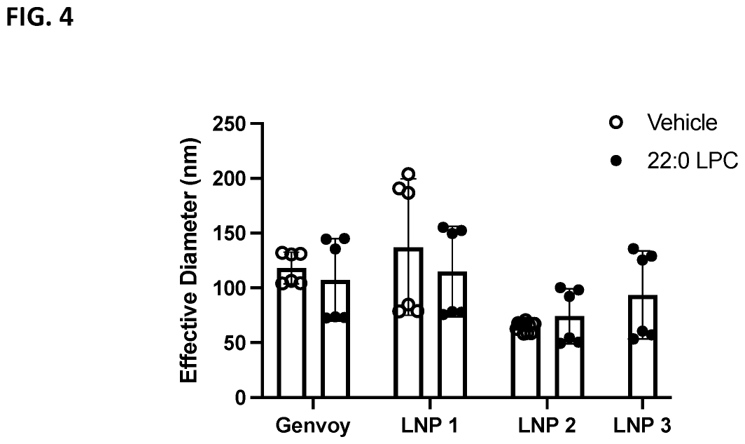

LNPs formulations were sized using dynamic light scattering (DLS) to determine their mean effective diameters. Two LNP batches of each formulation were prepared, and three sizing measurements were taken per batch. The LNPs ranged from 50 to 200 nm in diameter depending upon the formulation and the addition of 22:0 Lyso PC (FIG. 4). The addition of 22:0 Lyso PC did not appear to greatly impact the effective diameter of the LNPs in any of the formulations tested.

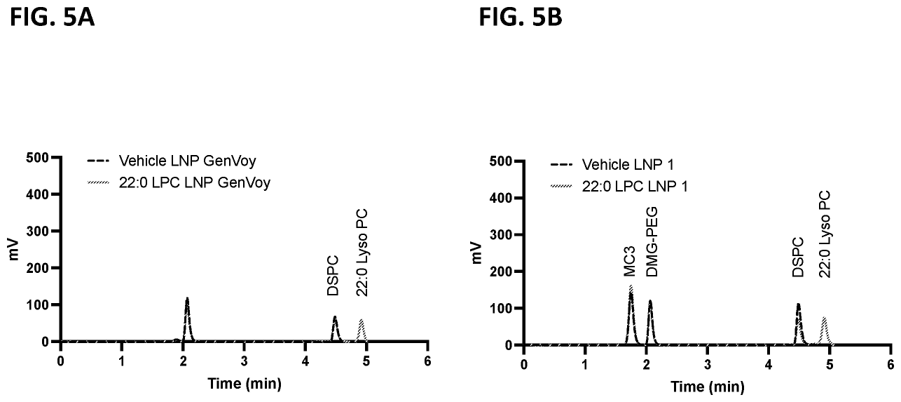

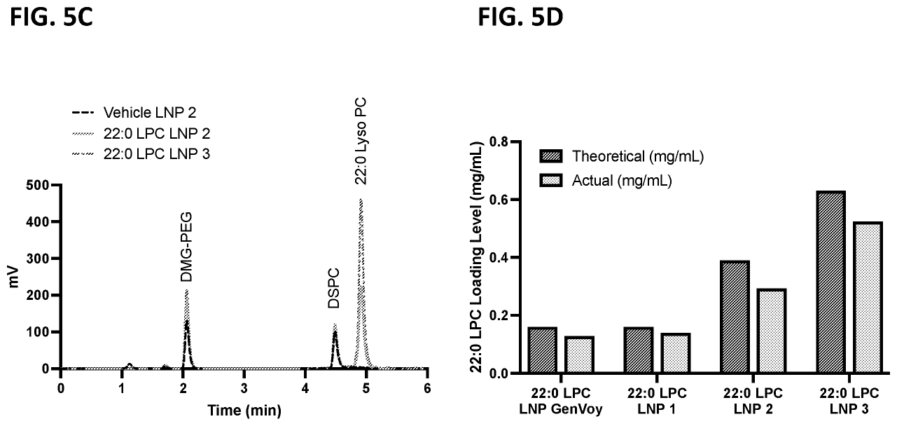

The four LNP formulations tested started with differing 22:0 LPC molar ratio inputs. In order to determine whether modulating the input ratio impacted the loading level of 22:0 Lyso PC in LNPs, the quantity of 22:0 Lyso PC present in LNPs was assessed using HPLC. LNP preparations were lyophilized and then dissolved in a mixture of ethanol and water for quantification. Samples were compared to a standard curve of 22:0 Lyso PC prepared using the same dissolution conditions. 22:0 Lyso PC was successfully detected in preparations where it was included in the starting input and not detected in their corresponding empty vehicle controls (FIG. 5A-5C). Assuming 100% efficiency of 22:0 Lyso PC incorporation into LNPs, theoretical values of lipid incorporation were calculated. The actual amounts of 22:0 Lyso PC measured via HPLC were within 75% of theoretical values (FIG. 5D). As the input of 22:0 Lyso PC increased, the measured 22:0 Lyso PC loaded into the LNPs also increased. This indicates that 22:0 Lyso PC loading in LNPs can be tuned depending on the amount of 22:0 Lyso PC needed for a particular application.

### Example 3: Hyperactivation of Human Dendritic Cells with a TLR7/8 Agonist in Combination with Lipid Nanoparticles Comprising a Lysophosphatidylcholine (LPC)

This example describes the hyperactivation of human monocyte-derived dendritic cells (moDCs) with a TLR7/8 agonist in combination LNPs loaded with a hyperactivating lipid (e.g., 22:0 LYSO PC).

### Materials and Methods

Human monocytes were isolated from Leukopaks purchased from Miltenyi Inc. (San Jose, CA) using the StraightFrom Leukopak CD14 microbead kit according to the manufacturer's instructions. Monocytes were then aliquoted and frozen in fetal bovine serum containing 10% dimethyl sulfoxide. For studies with monocyte-derived dendritic cell (moDC) cultures, monocytes were thawed and cultured in RPMI medium containing 10% FBS, 50 units/mL penicillin, 50 mg/mL streptomycin, 2 mM L-glutamine, 1 mM sodium pyruvate, 50 mM beta-mercaptoethanol, 10 mM HEPES, and Gibco MEM non-essential amino acids (R10 media). To differentiate monocytes into moCs, recombinant human GM-CSF (50 ng/ml) and IL-4 (25 ng/ml) were added to R10 media. Cells were cultured for 6 days with GM-CSF and IL-4, with an additional cell feeding with R10 media containing GM-CSF and IL-4 on day 3.

Six days after differentiation, moDC were collected and counted. Cells were plated into 96-well flat-bottom plates at 1×10⁵ cells/well. Cells were treated with or without 1 μg/mL R848 (final) and with or without a hyperactivating lipid (or vehicle control). Hyperactivity induced by LNPs was measured using two assays. The CellTiter-Glo assay (Promega) detects ATP as a measure of cell viability. The IL-1β Lumit assay (Promega) measures IL-1β cytokine present in the moDC cell culture supernatant. Experimental conditions were tested in triplicate and the mean result from one donor was plotted. Data represent results from six human donor samples tested across two experiments.

### Results

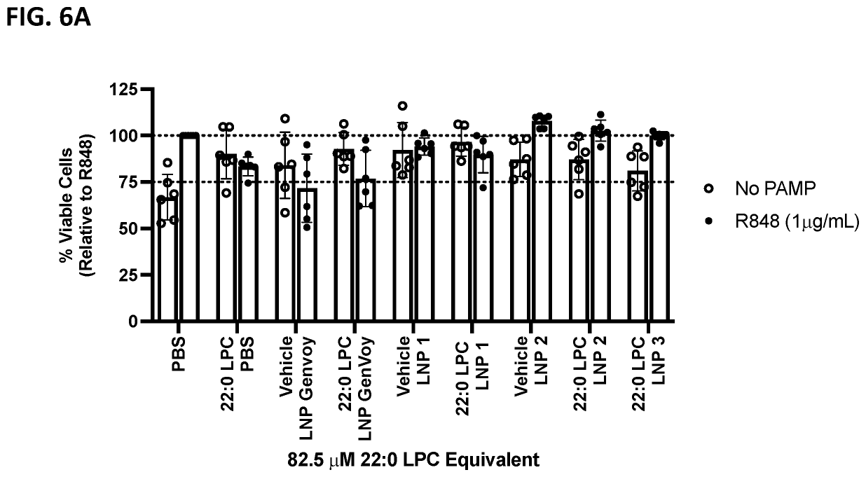

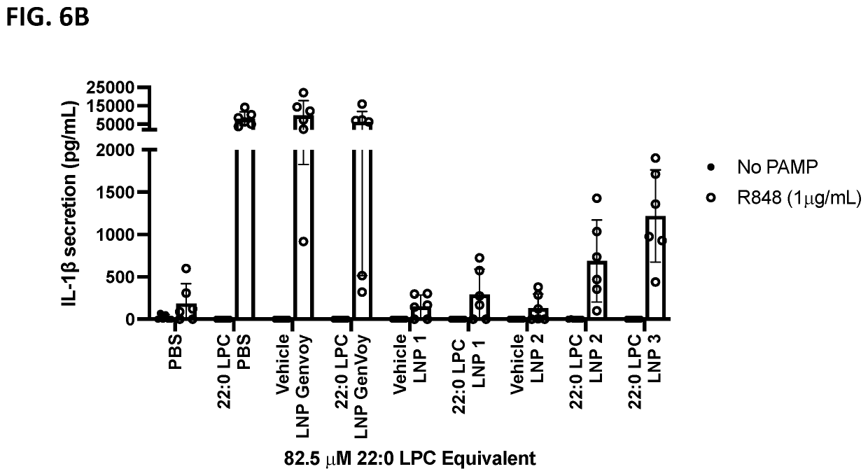

Hyperactive moDCs retain cell viability while producing IL-1β, an important cytokine in the generation and re-activation of long-lived memory T cells. The ability of the LNPs, which were prepared as described in Example 2, to hyperactivate human moDCs was tested. For comparison, 22:0 Lyso PC was also simply resuspended in PBS media, which renders it as a large, insoluble, flaky material. When cell viability was measured using the ATP quantification assay, most experiment conditions had negligible effect on cell viability (FIG. 6A). The most notable exception were conditions where moDCs were treated with LNPs made with GenVoy-ILM, which had a mean viability below 75% (relative to an R848-only treatment). The Gen Voy-ILM LNPs induced IL-1β production, regardless of whether or not 22:0 Lyso PC was included in the formulation (FIG. 6B). Given the decreased cell viability, the Gen Voy-ILM LNPs are presumed to have caused cell death, so IL-1β release may not be a result of moDC hyperactivation.

When cells were dosed with 22:0 Lyso PC resuspended directly in PBS, IL-1β was produced, with a cost in cell viability (FIG. 6A). In addition, the large flake format of 22:0 Lyso PC in PBS resulted in inconsistent dosing of moDCs, so replicates when using moDCs derived from a donor were variable. In comparison, LNP formulations had more consistent IL-1β responses. 22:0 Lyso PC LNPs induced IL-1β secretion above background measurements from their corresponding empty LNP vehicle controls (FIG. 6B). Interestingly, despite dosing 82.5 μM of 22:0 Lyso PC across the different formulations, an increasing amount of IL-1β was produced from cells that were treated with fewer LNPs loaded with more 22:0 Lyso PC per LNP. The formulations containing 10% 22:0 Lyso PC (GenVoy and LNP1) did not have increased immunogenicity compared to their vehicle controls. In contrast, formulations containing 25% or 40% 22:0 Lyso PC did induce higher levels of IL-1β secretion in comparison to their vehicle controls, with LNPs containing 40% 22:0 Lyso PC inducing the highest levels of IL-1β secretion. These data indicate that the given payload of 22:0 Lyso PC per LNP is an important contributing factor to moDC hyperactivation. The equivalent amount of 22:0 Lyso PC spread across more LNPs is less efficient at hyperactivation.

Taken together, the data demonstrate that 22:0 Lyso PC can be incorporated into LNPs. This new formulation method is contemplated to be clinically meaningful because it allows for fine tuning of the amount of 22:0 Lyso PC incorporated into a particle, which can have a significant impact on the efficiency of hyperactivation. Although hyperactivating, 22:0 Lyso PC in PBS cannot provide the same level of tunability. In addition, another concern with preparing 22:0 Lyso PC in PBS is that very large visible particles form that are not evenly distributed in solution. The unevenly distributed, large particles, are contemplated to present challenges for accurate dosing. Also, very large particles are likely to limit biodistribution of 22:0 Lyso PC in vivo, as the particulates that are visible to the eye are much larger than a cell. It is possible that the large particles could be sequestered by the immune system when delivered in vivo, which could limit the ability of 22:0 Lyso PC to reach dendritic cells for hyperactivation.

### Example 4: Hyperactivation of Murine Dendritic Cells with a TLR7/8 Agonist in Combination with Lipid Nanoparticles Comprising a Lysophosphatidylcholine (LPC)

This example describes the hyperactivation of murine bone marrow-derived dendritic cells (BMDCs) with a TLR7/8 agonist in combination lipid nanoparticles (LNPs) loaded with a hyperactivating lipid (e.g., 22:0 Lyso PC).

### Materials and Methods

LNP Synthesis. LNPs were prepared by combining the following components with or without 1-behenoyl-2-hydroxy-sn-glycero-3-phosphocholine
(CAS Registry No. 125146-65-8, referred to herein as “22:0 Lyso PC”) (Avanti):
   1,2-distearoyl-sn-glycero-3-phosphocholine
(CAS Registry No. 816-94-4, referred to herein as “DSPC”) (Avanti);
   cholesterol (Sigman); and
   1,2-dimyristoyl-rac-glycero-3-methoxypolyethylene glycol-2000
(CAS Registry No. 160743-62-4, referred to herein as “DMG-PEG2000”) (Avanti).
LNPs were either prepared without 22:0 Lyso PC, or loaded with 20% or 40% molar ratios of 22:0 Lyso PC to determine if the 22:0 Lyso PC loading could be intentionally varied. Lipid nanoparticles (LNPs) were synthesized using the NanoAssemblr Ignite instrument (Precision Nanosystems). Lipids were first dissolved in ethanol and then according to the molar percentages shown in Table 4-1 to reach a total lipid concentration of 12.5 mM. Lipids in ethanol were combined with sodium citrate buffer, pH 4 at a 1:3 volumetric ratio, at a flow rate of 12 mL/min. LNPs were washed in 10 volumes of phosphate buffered saline (PBS), pH 7.4 to remove residual ethanol, and then concentrated using Amicon 10K MWCO centrifugal filters.

LNP Characterization. Loading of 22:0 Lyso PC into LNPs was assessed using HPLC. LNPs in PBS were frozen at −20° C. until quantified. LNPs were dissolved by adding 1 part ethanol to the LNPs in PBS. A seven point standard curve of 22:0 Lyso PC was prepared in 1:1 ethanol: PBS added to match sample preparation. Standards and samples were filtered through a 0.45 μm filter prior to running on the HPLC. HPLC quantification was performed on using an Agilent 1260 Infinity II HPLC equipped with a 1260 Infinity II Evaporative Light Scattering Detector. A Luna 5 μm NH2 100 Å, 150X4.6 mm LC Column (Phenomenex) with a column temperature of 30° C. was used to detect samples. Two eluents were used: A, 100% water; and B, 100% acetonitrile. An initial mobile phase composed of 5%/95% A/B was used to load the column, with a gradient reaching 24%/76% A/B after 2.5 min. A more shallow gradient was used from 2.5 to 6 minutes, with A/B slowly reaching 25%/75% during that time frame. A post time of 3 min was used to return the gradient to starting conditions prior to the next sample run. The flow rate was set to 1 mL/min, and the injection volume was 2.5 μL for samples and standards. The evaporative light scattering detector (ELSD) used an evaporator temperature of 50° C., a nebulizer temperature of 30° C., and a gas flow rate of 0.9 standard liters per minute. Agilent CDS 2.6 software was used for HPLC instrument control, data acquisition, and processing.

The size of the LNPs was assessed using dynamic light scattering (DLS) on the NanoBrook Omni (Brookhaven) device. LNPs were diluted 1:10 in PBS before running on the DLS. Three 90 second measurements were recorded for each sample. The size of 22:0 Lyso PC in PBS was assessed using a Mastersizer 3000 (Malvern) equipped with a Hydro SV small volume dispersion unit set to a spin speed of 1500 rpm. Five readings of 5 seconds were recorded for each sample.

Murine bone marrow-derived FLT3L-DCs generation. Leg femur and tibia were removed from mice, cut with scissors, and flushed into sterile tubes. A bone marrow suspension was treated with ACK Lysis Buffer for 1 minute, then passed through a 40 μm cell strainer. Cells were counted and resuspended in media consisting of complete IMDM containing 10% FBS, penicillin and streptomycin, and supplements of L-glutamine and sodium pyruvate (I10). Cells were then plated at 8×10⁶ bone marrow cells per well in a P12 plate. Recombinant mouse FLT3L (Miltenyi) was added to cultures at 200 ng/mL. Differentiated cells were used for subsequent assays on day 8. The efficiency of differentiation was monitored by flow cytometry using a BD Symphony A3, and CD11c⁺MHC-II⁺ cells were routinely above 80% of living cells. For each experiment, 5 to 15 mice were used to generate bone marrow-derived dendritic cells (BMDCs).

Hyperactivation of murine bone marrow-derived FLT3L-DCs. BMDCs were harvested on day 8 post differentiation, washed with PBS and re-plated in FLT3L-containing complete IMDM media (110) at a concentration of 2×10⁵ cells/mL. Cells were cultured in the presence or absence of 1 μg/mL R848 then treated with or without 22:0 Lyso PC in PBS or 22:0 Lyso PC LNPs at 82 μM. Forty-eight hours post stimulation, supernatants were collected for cytokine measurement. Viability was measured using the CellTiter-Glo assay (Promega), which measures ATP content from cells. Fifty microliters of CellTiter-Glo reagent were added to 50 μL of cells. Luminescence was quantified on a SpectraMax M5e plate reader using an integration time of 500 milliseconds. Viability data were set relative to control conditions where cells were treated with R848. IL-1β and IL-6 cytokine secretion were measured using sandwich ELISAs (Invitrogen).

Quantification of Cell Viability. Cell viability was assessed by quantifying the presence of ATP as an indicator of metabolically active cells using the CellTiter-Glo Luminescent Cell Viability Assay (Promega). Metabolic activity was assessed following the manufacturer's instructions. The CellTiter-Glo reagent was mixed with the cell pellets and remaining supernatant, and transferred to a white, opaque 96-well plate. Luminescence was measured on all wavelengths on a Spectramax M5e plate reader (Molecular Devices) using an integration time of 500 ms. Percent viability was calculated relative to R848 treated DCs.

Quantification of IL-1β and IL-6 Secretion. IL-1β and IL-6 secretion was assessed using the ELISA Mouse IL-1β and IL-6 kits (Invitrogen). ELISAs were performed according to the manufacturer's instructions. Absorbance was measured at 450 nm, with a 570 nm correction, using a Spectramax M5e plate reader (Molecular Devices). To determine IL-1β and IL-6 concentrations in supernatants, sample IL-1β or IL-6 concentrations were interpolated using a standard curve via 4PL analysis on GraphPad Prism 9 (GraphPad Software). The interpolated results of samples were then adjusted for any dilutions made to the supernatant.

Hyperactivation of FLT3L-DCs for Migration Assay. Murine bone marrow-derived dendritic cells (BMDCs) were harvested on day 8 post differentiation, washed with PBS and re-plated in FLT3L-containing 110 at a concentration of 10×10⁶ cells/mL. For hyperactivation, 500 μl of R848 was added at a final concentration of 1 μg/mL, and 500 μL of lipids (22:0 Lyso PC prepared in PBS or 22:0 Lyso PC LNPs) at a final concentration of 82 μM. Cells were incubated for 24 hours at 37° C. on a tube rotator. Twenty-four hours post-stimulation, cells were washed with PBS and stained with CFSE (1:1000) for 30 minutes at 37° C. in the dark. DCs were then counted and 1×10⁶ cells were injected subcutaneously (SC) in 100 μL per mouse. 24 hours post-injection, the skin draining lymph nodes (dLN) were dissected from the side of injection. A single cell suspension was prepared, and cells were stained in PBS with Live/Dead Fixable dye (ThermoFisher) for 20 minutes at 4° C. Cells were then washed again and stained for 20 minutes at 4° C. in MACS buffer (PBS with 1% FCS and 2 mM EDTA) containing the following fluorescently conjugated antibodies: anti-CD11c, and anti-I-A/I-E (MHC-II). To determine the absolute number of CD11c⁺ MHC-II⁺ among living cells, countBright counting beads (ThermoFisher) were used, following the manufacturer's protocol. Data were acquired on a BD FACS Symphony (Becton-Dickenson). Data were analyzed using FlowJo software (Tree Star). Four mice were used for each experimental group.

### Results

22:0 Lyso PC can be loaded into LNPs. 22:0 Lyso PC was efficiently incorporated into LNPs comprising DSPC, cholesterol and DMG-PEG2000. Using the LNP synthesis process described above, 87.4% of the 22:0 Lyso PC that was added during synthesis was recovered from LNPs and detected by HPLC as shown in Table 4-1.

TABLE 4-1
Percent Molarity of LNP Components

| | | | Encapsulation |
|---|---|---|---|
| Lipid | Empty LNP | 22:0 Lyso PC LNP | Efficiency (%) |
| DSPC | 50.0% | 10.0% | |
| Cholesterol | 48.5% | 48.5% | |
| DMG-PEG2000 | 1.5% | 1.5% | |
| 22:0 Lyso PC | 0.0% | 40.0% | 87.4 ± 0.1 |

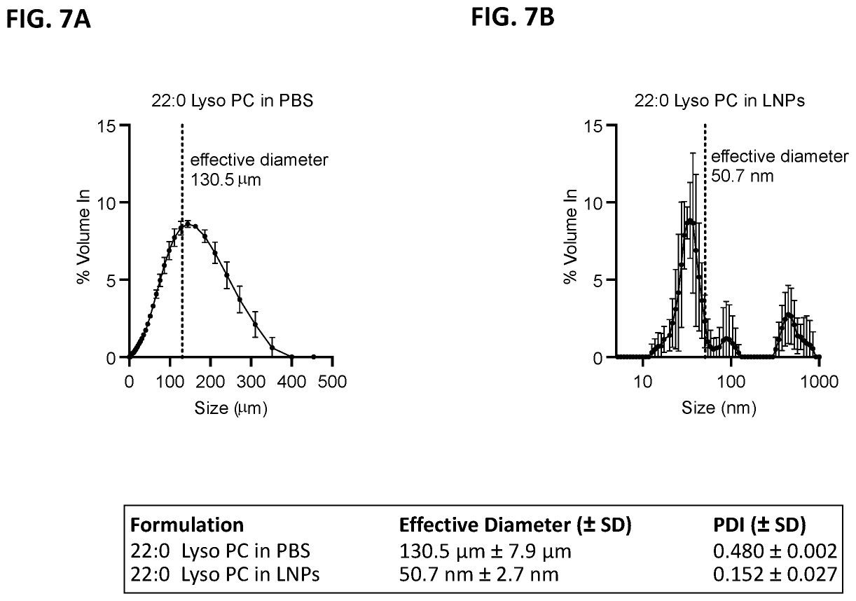

22:0 Lyso PC in LNP preparations are more uniform. One concern with preparing 22:0 Lyso PC in PBS is that the 22:0 Lyso PC is insoluble, and therefore results in large particles that are not evenly distributed in solution. The particles are on the order of 130 μm in diameter (FIG. 7A) with a large polydispersity index, indicating a wide range in particle sizes. This particle size is too large to be taken up by cells (e.g., phagocytes) as it is roughly 10-fold larger than the size of a cell. As a result, these large particles may not be to reach dendritic cells (or other cells of interest) in vivo. In contrast, when 22:0 Lyso PC is incorporated into LNPs, the LNPs are on the order of ˜50 nm in size, with the largest of the particles (that have not been filtered) less than 1 μm in diameter (FIG. 7B). In addition, the polydispersity index (PDI) of these particles is much smaller, indicating a more uniform suspension. At this size, the LNPs can be were readily taken up by cells. Thus, 22:0 Lyso PC is expected to be more bioavailable in vivo. The uniformity in 22:0 Lyso PC distribution in an LNP suspension is expected to translate into more repeatable and accurate dosing levels.

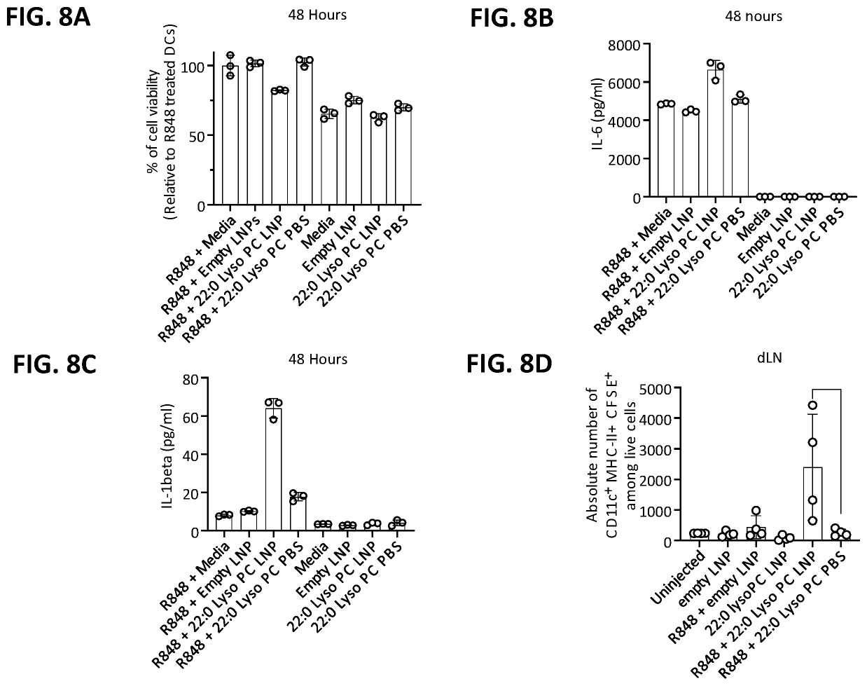

22:0 Lyso PC LNPs induce IL-1β secretion from murine DCs in vitro. FLT3L-DCs were stimulated with media alone, empty LNPs, or 82 μM of 22:0 Lyso PC in PBS or loaded into LNPs. Alternatively, FLT3L-DCs were treated with R848 at 1 μg/ml in combination with empty LNPs, 22:0 Lyso PC LNPs, or 22:0 Lyso PC in PBS. 48 hours post stimulation, cell supernatants were harvested for ELISA and cell viability was measured by cell Titer Glow assay, which measure the levels of ATP release from cells. When cells were treated with R848 in combination with the hyperactivating lipid formulation (22:0 Lyso PC in PBS or LNP), or in combination with empty LNP, FLT3L DCs were viable as revealed by the percent of cell viability compared to R848 alone (FIG. 8A). In addition, the stimulation with R848 in combination with empty LNPs or 22:0 Lyso PC induced high levels of the pro-inflammatory cytokine IL-6 regardless of whether 22:0 Lyso PC was formulated in PBS or LNP (FIG. 8B). Furthermore, while R848 and 22:0 Lyso PC in PBS did not induce IL-1β secretion from viable cells, the treatment of DCs with R848 in combination with 22:0 Lyso PC in LNPs did induce IL-1β secretion from viable cells (FIG. 8C), indicating that 22:0 Lyso PC in LNP induce DC hyperactivation and has an advantage over 22:0 Lyso in PBS.

22:0 Lyso PC LNPs induce DC hypermigration in vivo. Another hallmark of hyperactivation is the ability of the hyperactivating lipid to induce DC hypermigration from the skin to the draining lymph node (dLN). To assess if 22:0 Lyso PC in LNPs could induce DC migration, FLT3L-DCs were incubated on a tube rotator overnight with empty LNPs, 22:0 Lyso PC in LNPs, or R848 in combination with empty LNPs, 22:0 Lyso PC in LNPs or 22:0 Lyso PC in PBS. The next day, cells were washed and stained with CFSE. 1×10⁶ cells were injected subcutaneously per mouse on the right back. 24 hours post-injection, the dLN were harvested and a single cell suspension was prepared. dLN from mice that were not injected were used as a negative control. Cells were stained with Live/Dead to identify living cells, CD11c and MHC-II. The percentage of CFSE⁺, CD11c⁺ MHC-II⁺ DCs was measured by flow cytometry. As expected, DCs treated with R848 in combination with empty LNP or 22:0 Lyso PC LNP alone did not induce any DC migration from the skin to the dLN (FIG. 8D). Similarly, DC treated with R848 in combination with empty LNP or R848 in combination with 22:0 Lyso PC in PBS did not induce DC migration to the dLN. Interestingly, DCs that were treated with R848 in combination with 22:0 Lyso PC in LNPs enhanced migration of DC to the dLN (FIG. 8D).

These data demonstrate that 22:0 Lyso PC-containing LNPs is a superior hyperactivating lipid formulation compared to 22:0 Lyso PC in an aqueous buffer such as PBS. DCs treated with 22:0 Lyso PC delivered in LNPs showed an increase in IL-1β secretion and an increase in migration to draining lymph nodes, compared to LNPs devoid of 22:0 Lyso PC and to 22:0 Lyso PC formulated in PBS. Thus, 22:0 Lyso PC delivered in LNPs is contemplated to result in more potent de novo T cell (and in particular, memory T cell) generation when delivered with antigen in vivo than antigen delivered with 22:0 Lyso PC in PBS (or with LNPs devoid of 22:0 Lyso PC).

We claim:

1. A composition comprising an isolated lysophosphatidylcholine (LPC) with a single acyl chain, at least one further lipid, and a TLR7/8 agonist, wherein
the acyl chain is a C13-C22 acyl chain or a C13-C24 acyl chain,
the at least one further lipid is selected from the group consisting of an ionizable lipid, a cationic lipid, a further phospholipid, a pegylated lipid, a structural lipid, and mixtures thereof, and
the LPC and the at least one further lipid are part of a lipid nanoparticle (LNP).

2. The composition of claim 1, wherein the acyl chain is a C18-C22 acyl chain or a C21-C24 acyl chain.

3. The composition of claim 1 or claim 2, further comprising an antigen.

4. The composition of any one of claims 1-3, further comprising dendritic cells.

5. A composition comprising an isolated lysophosphatidylcholine (LPC) with a single acyl chain, at least one further lipid, and an antigen, wherein
the acyl chain is a C21-C24 acyl chain,
the at least one further lipid is selected from the group consisting of an ionizable lipid, a cationic lipid, a further phospholipid, a pegylated lipid, a structural lipid, and mixtures thereof, and
the LPC and the at least one further lipid are part of a lipid nanoparticle (LNP).

6. The composition of claim 5, further comprising dendritic cells.

7. The composition of claim 5 or claim 6, further comprising a TLR7/8 agonist.

8. A composition comprising an isolated lysophosphatidylcholine (LPC) with a single acyl chain, at least one further lipid, and dendritic cells, wherein
the acyl chain is a C21-C24 acyl chain,
the at least one further lipid is selected from the group consisting of an ionizable lipid, a cationic lipid, a further phospholipid, a pegylated lipid, a structural lipid, and mixtures thereof, and
the LPC and the at least one further lipid are part of a lipid nanoparticle (LNP).

9. The composition of claim 8, further comprising a TLR7/8 agonist.

10. The composition of claim 8 or claim 9, further comprising an antigen.

11. The composition of any one of claims 1-10, wherein the acyl chain is a C22 acyl chain.

12. The composition of any one of claims 1-11, wherein the acyl chain is fully saturated.

13. The composition of any one of claims 1-12, wherein the LPC comprises 1-behenoyl-2-hydroxy-sn-glycero-3-phosphocholine [LPC(22:0)].

14. The composition of any one of claims 1-13, wherein the TLR7/8 agonist is a small molecule with a molecule weight of 900 daltons or less.

15. The composition of claim 14, wherein the TLR7/8 agonist comprises an imidazoquinoline compound.

16. The composition of claim 15, wherein the TLR7/8 agonist comprises resiquimod (R848).

17. The composition of claim 14 or claim 15, wherein the TLR7/8 agonist does not inhibit NLR family pyrin domain containing 3 (NLRP3).

18. The composition of claim 13, wherein the LPC comprises LPC(22:0), and the TLR7/8 agonist comprises resiquimod (R848).

19. The composition of any one of claims 1-18, wherein the antigen is present in a biological sample obtained from an individual.

20. The composition of claim 19, wherein the biological sample comprises biopsy tissue.

21. The composition of claim 19, wherein the biological sample comprises cells.

22. The composition of claim 19, wherein the biological sample does not comprise cells.

23. The composition of claim 19, wherein the biological sample comprises pus from an abscess.

24. The composition of any one of claims 1-23, wherein the antigen comprises a proteinaceous antigen.

25. The composition of claim 24, wherein the antigen comprises a tumor antigen.

26. The composition of claim 25, wherein the tumor antigen comprises a synthetic or recombinant neoantigen.

27. The composition of claim 26, wherein the tumor antigen comprises a tumor cell lysate.

28. The composition of claim 24, wherein the antigen comprises a microbial antigen and the microbial antigen comprises one or more of a viral antigen, a bacterial antigen, a protozoan antigen, and a fungal antigen.

29. The composition of claim 28, wherein the microbial antigen comprises a purified or recombinant surface protein.

30. The composition of claim 28, wherein the microbial antigen comprises an inactivated, whole virus.

31. The composition of any one of claims 1-30, wherein the composition comprises liposomes.

32. The composition of any one of claims 1-31, wherein the composition does not comprise lipopolysaccharide (LPS) or monophosphoryl lipid A (MPLA).

33. The composition of any one of claims 1-32, wherein the composition does not comprise oxidized 1-palmitoyl-2-arachidonoyl-sn-glycero-3-phosphorylcholine (oxPAPC) or a species of oxPAPC.

34. The composition of claim 33, wherein the composition does not comprise 2-[[(2R)-2-[(E)-7-carboxy-5-hydroxyhept-6-enoyl]oxy-3-hexadecanoyloxypropoxy]-hydroxyphosphoryl]oxyethyl-trimethylazanium (HOdiA-PC), [(2R)-2-[(E)-7-carboxy-5-oxohept-6-enoyl]oxy-3-hexadecanoyloxypropyl] 2-(trimethylazaniumyl) ethyl phosphate (KOdiA-PC), 1-palmitoyl-2-(5-hydroxy-8-oxo-octenoyl)-sn-glycero-3-phosphorylcholine (HOOA-PC), 2-[(2R)-2-[(E)-5,8-dioxooct-6-enoyl]oxy-3-hexadecanoyloxypropoxy]-hydroxyphosphoryl]oxyethyl-trimethylazanium (KOOA-PC), [(2R)-3-hexadecanoyloxy-2-(5-oxopentanoyloxy) propyl] 2-(trimethylazaniumyl) ethyl phosphate (POVPC), [(2R)-2-(4-carboxybutanoyloxy)-3-hexadecanoyloxy propyl] 2-(trimethylazaniumyl) ethyl phosphate (PGPC), [(2R)-3-hexadecanoyloxy-2-[4-[3-[(E)-[2-[(Z)-oct-2-enyl]-5-oxocyclopent-3-en-1-ylidene]methyl]oxiran-2-yl]butanoyloxy]propyl] 2-(trimethylazaniumyl) ethyl phosphate (PECPC), [(2R)-3-hexadecanoyloxy-2-[4-[3-[(E)-[3-hydroxy-2-[(Z)-oct-2-enyl]-5-oxocyclopentylidene]methyl]oxiran-2-yl]butanoyloxy]propyl] 2-(trimethylazaniumyl) ethyl phosphate (PEIPC) and/or 1-palmitoyl-2-azelaoyl-sn-glycero-3-phosphocholine (PAzePC).

35. The composition of any one of claims 1-34, further comprising an adjuvant, wherein the adjuvant comprises an aluminum salt adjuvant, a squalene-in-water emulsion, a saponin, or combinations thereof.

36. A pharmaceutical formulation comprising the composition of any one of claims 1-35 and a pharmaceutically acceptable excipient.

37. A method for production of hyperactivated dendritic cells, the method comprising contacting the dendritic cells with an effective amount of a composition comprising an isolated lysophosphatidylcholine (LPC) with a single C13-C22 acyl chain or a C13-C24 acyl chain, at least one further lipid, and a TLR7/8 agonist, for production of hyperactivated dendritic cells, wherein
the hyperactivated dendritic cells secrete IL-1beta without undergoing pyroptosis,
the at least one further lipid is selected from the group consisting of an ionizable lipid, a cationic lipid, a further phospholipid, a pegylated lipid, a structural lipid, and mixtures thereof, and
the LPC and the at least one further lipid are part of a lipid nanoparticle (LNP).

38. The method of claim 37, wherein the dendritic cells are contacted ex vivo with the composition of any one of claims 1-35 or the formulation of claim 36.

39. The method of claim 37, wherein the dendritic cells are contacted in vivo with the formulation of claim 36.

40. A pharmaceutical formulation comprising at least 103, 104, 105 or 106 of the hyperactivated dendritic cells produced by the method of claim 38, and a pharmaceutically acceptable excipient.

41. A method of stimulating an immune response against an antigen, comprising administering an effective amount of the formulation of claim 36 to an individual in need thereof to stimulate the immune response against the antigen.

42. A method of treating cancer, comprising administering an effective amount of the formulation of claim 36 to an individual in need thereof to treat the cancer.

43. A method of inhibiting abnormal cell proliferation, comprising administering an effective amount of the formulation of claim 36 to an individual in need thereof to inhibit abnormal cell proliferation.

44. A method of treating an infectious disease, comprising administering an effective amount of the formulation of claim 36 to an individual in need thereof to treat the infectious disease.

45. Use of the formulation of claim 36 for inducing an immune response against the antigen in an individual in need thereof.

46. Use of the formulation of claim 36 for inducing an anti-tumor immune response in an individual in need thereof, wherein the individual is or was tumor-bearing.

47. Use of the formulation of claim 36 for inducing an anti-microbe immune response in an individual in need thereof, wherein the individual is infected with the microbe or has not been exposed to the microbe.

48. The composition, formulation, method or use of any one of claims 19-47, wherein the individual is a mammalian subject.

49. The composition, formulation, method or use of any one of claims 19-47, wherein the individual is a human subject.

50. A method of preparing an immunogenic composition, the method comprising:
a) obtaining a tumor cell-enriched suspension from a tumor;
b) lysing cells from the tumor cell-enriched suspension to obtain a tumor cell lysate; and
c) contacting the tumor cell lysate with a composition comprising an isolated lysophosphatidylcholine (LPC) having a single acyl chain, at least one further lipid, and a toll-like receptor 7/8 (TLR7/8) agonist, to obtain the immunogenic composition, wherein
the acyl chain is a C13-C22 acyl chain or a C13-C24 acyl chain,
the at least one further lipid is selected from the group consisting of an ionizable lipid, a cationic lipid, a further phospholipid, a pegylated lipid, a structural lipid, and mixtures thereof, and
the LPC and the at least one further lipid are part of a lipid nanoparticle (LNP).

51. The method of claim 50, wherein step a) comprising depleting leukocytes from the tumor cell-enriched suspension, optionally wherein the leukocytes are depleted by negative selection using an anti-CD45 antibody.

52. The method of claim 50 or claim 51, wherein the cells are lysed in step b) by one or more freeze-thaw cycles.

53. The method of any one of claims 50-52, wherein the acyl chain is a fully saturated C18-C22 acyl chain or a fully saturated C18-C24 acyl chain.

54. The method of claim 53, wherein the LPC comprises 1-behenoyl-2-hydroxy-sn-glycero-3-phosphocholine [LPC(22:0)].

55. The method of any one of claims 50-54, wherein the TLR7/8 agonist is a small molecule with a molecule weight of 900 daltons or less.

56. The method of claim 55, wherein the TLR7/8 agonist comprises an imidazoquinoline compound.

57. The method of claim 56, wherein the TLR7/8 agonist comprises resiquimod (R848).

58. The method of claim 55 or claim 56, wherein the TLR7/8 agonist does not inhibit NLR family pyrin domain containing 3 (NLRP3).

59. The method of claim 54, wherein the LPC comprises LPC(22:0), and the TLR7/8 agonist comprises resiquimod (R848).

60. The method of any one of claims 50-59, further comprising before step a) obtaining a sample from the tumor from a mammalian subject with cancer and preparing the suspension of cells from the sample.

61. An immunogenic composition prepared by the method of any one of claims 50-60.

62. A method of eliciting an anti-cancer immune response, the method comprising:
administering to a mammalian subject with cancer an effective amount of the immunogenic composition of claim 61.

63. The method of claim 62, wherein the anti-cancer immune response comprises cellular immune response.

64. The method of claim 63, wherein the anti-cancer immune response comprises cancer antigen-induced IL-1beta secretion and/or activation of CD8+T lymphocytes.

65. The method of any one of claims 62-64, wherein the cancer is a non-hematologic cancer.

66. The method of claim 65, wherein the non-hematologic cancer is a carcinoma, a sarcoma, or a melanoma.

67. The method of any one of claims 62-64, wherein the cancer is a lymphoma.

68. A method of treating cancer, the method comprising:
a) preparing an immunogenic composition comprising a tumor cell lysate, an isolated lysophosphatidylcholine (LPC) having a single acyl chain, at least one further lipid, and a toll-like receptor 7/8 (TLR7/8) agonist, wherein
the tumor cell lysate is or has been prepared from a sample of a tumor obtained from the mammalian subject with cancer,
the acyl chain is a C13-C22 acyl chain or a C13-C24 acyl chain,
the at least one further lipid is selected from the group consisting of an ionizable lipid, a cationic lipid, a further phospholipid, a pegylated lipid, a structural lipid, and mixtures thereof, and
the LPC and the at least one further lipid are part of a lipid nanoparticle (LNP); and
b) administering to the subject an effective amount of the immunogenic composition.

69. The method of any one of claims 62-68, wherein the acyl chain is a fully saturated C18-C22 acyl chain or a fully saturated C18-C24 acyl chain.

70. The method of claim 68, wherein the LPC comprises 1-behenoyl-2-hydroxy-sn-glycero-3-phosphocholine [LPC(22:0)].

71. The method of any one of claims 62-70, wherein the TLR7/8 agonist is a small molecule with a molecule weight of 900 daltons or less.

72. The method of claim 71, wherein the TLR7/8 agonist comprises an imidazoquinoline compound.

73. The method of claim 72, wherein the TLR7/8 agonist comprises resiquimod (R848).

74. The method of claim 70, wherein the LPC comprises 22:0 LPC, and the TLR7/8 agonist comprises resiquimod (R848).

75. The method of any one of claims 68-74, further comprising administering to the subject an effective amount of an additional therapeutic agent.

76. The method of claim 75, wherein the additional therapeutic agent comprises one or more of the group consisting of an immune checkpoint inhibitor, an antineoplastic agent, and radiation therapy.

77. A composition comprising an isolated lysophosphatidylcholine (LPC) with a single acyl chain, at least one further lipid, and a pathogen recognition receptor (PRR) agonist, wherein
the acyl chain is a C13-C22 acyl chain or a C13-C24 acyl chain,
the at least one further lipid is selected from the group consisting of an ionizable lipid, a cationic lipid, a further phospholipid, a pegylated lipid, a structural lipid, and mixtures thereof, and
the LPC and the at least one further lipid are part of a lipid nanoparticle (LNP).

78. The composition of claim 77, wherein the PRR agonist is an agonist of a toll-like receptor (TLR), a NOD-like receptor (NLR), a RIG-I-like receptor (RLR), or a C-type lectin receptor (CLR).

79. The composition of claim 77, Wherein the PRR agonist is an agonist of a cytosolic DNA sensor (CDS) or a stimulator of IFN genes (STING).

80. The composition of claim 77, wherein the PRR agonist comprises one or more of R848, TL8-506, LPS, Pam2CSK4, and ODN 2336.

81. The composition of any one of claims 77-80, further comprising an antigen.

82. The composition of any one of claims 77-81, further comprising dendritic cells.

83. A pharmaceutical formulation comprising the composition of any one of claims 77-82 and a pharmaceutically acceptable excipient.

84. A pharmaceutical formulation comprising an isolated lysophosphatidylcholine (LPC) with a single acyl chain, at least one further lipid, and a pharmaceutically acceptable excipient, wherein
the acyl chain is a C21-C24 acyl chain,
the at least one further lipid is selected from the group consisting of an ionizable lipid, a cationic lipid, a further phospholipid, a pegylated lipid, a structural lipid, and mixtures thereof, and
the LPC and the at least one further lipid are part of a lipid nanoparticle (LNP).

85. The pharmaceutical formulation of claim 83 or claim 84, wherein the acyl chain is a fully saturated C22 acyl chain.

86. The pharmaceutical formulation of claim 85, wherein the LPC comprises 1-behenoyl-2-hydroxy-sn-glycero-3-phosphocholine [LPC(22:0)].

87. A composition for hyperactivation of human dendritic cells, comprising an isolated lysophosphatidylcholine (LPC) with a single acyl chain, at least one further lipid, and a pathogen recognition receptor (PRR) agonist, wherein
the acyl chain is C22 acyl chain,
the at least one further lipid is selected from the group consisting of an ionizable lipid, a cationic lipid, a further phospholipid, a pegylated lipid, a structural lipid, and mixtures thereof,
the LPC and the at least one further lipid are part of a lipid nanoparticle (LNP), and
the composition is effective for achieving a higher level of dendritic cell hyperactivation than a comparator composition comprising PGPC in place of the LPC.

88. The composition of claim 87, wherein the higher level of dendritic cell hyperactivation comprises induction of IL-1beta secretion from the human dendritic cells in vitro at a level that is at least 2, 3 or 4 fold higher when contacted with the composition comprising the LPC and the PRR agonist than when contacted with the comparator composition comprising the PGPC and the PRR agonist, wherein the PRR agonist is LPS.

89. The composition of claim 88, wherein the concentration of the LPC and the concentration of the PGPC are the same concentration in a range of from about 10 μM to about 80 μM, and the LPS is present at a concentration of 1 μg/ml in both the composition and the comparator composition.

90. The composition of claim 88, wherein the higher level of dendritic cell hyperactivation comprises a lipid activity index for IL-1beta secretion from the human dendritic cells for the composition comprising the LPC and the PRR agonist that is at least 4, 5 or 6 fold higher in activity units than that of the comparator composition comprising the PGPC and the PRR agonist.

91. The composition, formulation, method or use of any one of claims 19-47, wherein the individual is a canine subject.

92. The composition, formulation, method or use of any one of claims 60-90, wherein the mammalian subject is a human patient.

93. The composition, formulation, method or use of any one of claims 60-90, wherein the mammalian subject is a non-human patient.

94. The composition, formulation, method or use of any one of claims 60-90, wherein the mammalian subject is a canine patient.

95. The composition, formulation, method or use of any one of claim 1-90 or 92, wherein the dendritic cells are human dendritic cells.

96. The composition, formulation, method or use of any one of claim 1-91 or 94, wherein the dendritic cells are canine dendritic cells.

97. The composition, formulation, method or use of claim 95 or claim 96, wherein the dendritic cells are present in a composition comprising peripheral blood mononuclear cells (PBMCs).

98. The composition, formulation, method or use of any one of claims 37-49 or claim 91, wherein the hyperactivated dendritic cells secrete one or both of IFNγ and TNFα.

99. The composition, formulation, method or use of any one of claims 1-98, wherein the at least one further lipid comprises one or both of a further phospholipid and a structural lipid, optionally wherein the further phospholipid comprises 1,2-distearoyl-sn-glycero-3-phosphocholine (DSPC), and the structural lipid comprises cholesterol.

100. The composition, formulation, method or use of claim 99, wherein the at least one further lipid comprises a pegylated lipid, optionally wherein the pegylated lipid comprises polyethylene glycol [PEG] 2000 dimyristoyl glycerol [DMG].

101. The composition, formulation, method or use of claim 99 or claim 100, wherein the at least one further lipid comprises an ionizable lipid, optionally wherein the ionizable lipid comprises 4-(dimethylamino)-butanoic acid, (10Z,13Z)-1-(9Z,12Z)-9,12-octadecadien-1-yl-10,13-nonadecadien-1-yl ester (DLin-MC3-DMA) or analogs or derivatives thereof.

102. A composition comprising a lipid nanoparticle (LNP), wherein the LNP comprises a first phospholipid, and at least one lipid selected from the group consisting of an ionizable lipid, a second phospholipid, a pegylated lipid, a structural lipid, and mixtures thereof, wherein the first phospholipid comprises a lysophosphatidylcholine (LPC) with a single acyl chain, and the acyl chain is a C13-C24 acyl chain.

103. A composition comprising a lipid nanoparticle (LNP), wherein the LNP comprises a first phospholipid, an ionizable lipid, a second phospholipid, a pegylated lipid, and a structural lipid, wherein the first phospholipid comprises a lysophosphatidylcholine (LPC) with a single acyl chain, and the acyl chain is a C13-C24 acyl chain.

104. The composition of any one of claims 1-103, wherein the ionizable lipid comprises:
i) 8-[(2-hydroxyethyl) [6-oxo-6-(undecyloxy) hexyl]amino]-octanoic acid, 1-octylnonyl ester (SM-102) or analogs or derivatives thereof; and/or 6-((2-hexyldecanoyl)oxy)-N-(6-((2-hexyldecanoyl)oxy) hexyl)-N-(4-hydroxybutyl) hexan-1-aminium (ALC-0315) or analogs or derivatives thereof; or
ii) (6Z,9Z,28Z,31Z)-heptatriaconta-6,9,28,31-tetraen-19-yl 4-(dimethylamino) butanoate (DLin-MC3-DMA) or analogs or derivatives thereof.

105. The composition of any one of claims 1-104, wherein the pegylated lipid is selected from the group consisting of a PEG-modified phosphatidyiethanolamine, a PEG-modified phosphatide acid, a PEG-modified ceramide, a PEG-modified dialkylamine, a PEG-modified diacylglycerol, a PEG-modified dialkylglyerol, and combinations thereof.

106. The composition of any one of claims 1-104, wherein the pegylated lipid comprises polyethylene glycol [PEG] 2000 dimyristoyl glycerol [DMG].

107. The composition of any one of claims 1-106, wherein the structural lipid is selected from the group consisting of cholesterol, fecosterol, sitosterol, ergosterol, campesterol, stigmasterol, brassicasterol, tomatidine, ursolic acid, alpha-tocopherol, and combinations thereof.

108. The composition of any one of claims 1-106, wherein the structural lipid comprises cholesterol.

109. The composition of any one of claims 1-108, wherein the further phosholipid or the second phospholipid comprises a hydrophilic head moiety selected from the group consisting of phosphatidyl choline, phosphatidyl ethanolamine, phosphatidyl glycerol, phosphatidyl serine, phosphatidic acid, 2-lysophosphatidyl choline, and sphingomyelin.

110. The composition of claim 1-108, wherein the further phosholipid or the second phospholipid comprises one or more fatty acid tail moieties selected from the group consisting of lauric acid, myristic acid, myristoleic acid, palmitic acid, palmitoleic acid, stearic acid, oleic acid, linoleic acid, alpha-linolenic acid, erucic acid, arachidic acid, arachidonic acid, phytanoic acid, eicosapentaenoic acid, behenic acid, docosapentaenoic acid, and docosahexaenoic acid.

111. The composition of any one of claims 1-108, wherein the further phosholipid or the second phospholipid is selected from the group consisting of
1,2-dilinoleoyl-sn-glycero-3-phosphocholine (DLPC),
1,2-dimyristoyl-sn-glycero-phosphocholine (DMPC),
1,2-dioleoyl-sn-glycero-3-phosphocholine (DOPC),
1,2-dipalmitoyl-sn-glycero-3-phosphocholine (DPPC),
1,2-distearoyl-sn-glycero-3-phosphocholine (DSPC),
1,2-diundecanoyl-sn-glycero-phosphocholine (DUPC),
1-palmitoyl-2-oleoyl-sn-glycero-3-phosphocholine (POPC),
1,2-di-O-octadecenyl-sn-glycero-3-phosphocholine,
1-oleoyl-2-cholesterylhemisuccinoyl-sn-glycero-3-phosphocholine,
1,2-dilinolenoyl-sn-glycero-3-phosphocholine,
1,2-diarachidonoyl-sn-glycero-3-phosphocholine,
1,2-didocosahexaenoyl-sn-glycero-3-phosphocholine,
1,2-dioleoyl-sn-glycero-3-phosphoethanola mine (DOPE),
1,2-diphytanoyl-sn-glycero-3-phosphoethanolamine,
1,2-distearoyl-sn-glycero-3-phosphoethanolamine,
1,2-dilinoleoyl-sn-glycero-3-phosphoethanolamine,
1,2-dilinolenoyl-sn-glycero-3-phosphoethanolamine,
1,2-diarachidonoyl-sn-glycero-3-phosphoethanolamine,
1,2-didocosahexaenoyl-sn-glycero-3-phosphoethanolamine,
1,2-dioleoyl-sn-glycero-3-phospho-rac-(1-glycerol) sodium salt (DOPG),
sphingomyelin, and
combinations thereof.

112. The composition of any one of claims 1-108, wherein the further phosholipid or the second phospholipid comprises 1,2-distearoyl-sn-glycero-3-phosphocholine (DSPC).

113. The composition of any one of claims 1-112, wherein the at least one further lipid comprises: i) a cationic lipid, and comprises or further comprises ii) a neutral or anionic lipid.

114. The composition of claim 113, wherein the cationic lipid comprises one or both of:
i) 1,2-di-O-octadecenyl-3-trimethylammonium propane (DOTMA) or analogs or derivatives thereof; and
ii) 1,2-dioleoyl-3-trimethylammonium propane (DOTAP) or analogs or derivatives thereof.

115. The composition of claim 113 or claim 114, wherein the neutral or anionic lipid comprises:
i) 1,2-di-(9Z-octadecenoyl)-sn-glycero-3-phosphoethanolamine (DOPE) or analogs or derivatives thereof; and/or
ii) cholesterol or analogs or derivatives thereof; and/or
iii) 1,2-dioleoyl-sn-glycero-3-phosphocholine (DOPC) or analogs or derivatives thereof.

116. The composition of any one of claims 102-115, wherein the acyl chain of the LPC is a C21-C24 acyl chain.

117. The composition of any one of claims 102-115, wherein the acyl chain of the LPC is a C22 acyl chain.

118. The composition of any one of claims 102-117, wherein the composition further comprises a TLR7/8 agonist.

119. The composition of claim 118, wherein the TLR7/8 agonist comprises an imidazoquinoline compound.

120. The composition of claim 119, wherein the TLR7/8 agonist comprises resiquimod (R848).

121. The composition of claim 119, wherein the LPC comprises LPC(22:0), and the TLR7/8 agonist comprises resiquimod (R848)

122. The composition of any one of claims 102-121, wherein the composition further comprises an antigen.

123. The composition of claim 122, wherein the antigen is a tumor antigen or a neoantigen.

124. The composition of claim 122, wherein the antigen is a microbioal antigen, optionally wherein the microbial antigen a viral antigen, a bacterial antigen, a protozoan antigen, or a fungal antigen.

125. The composition, formulation, method or use of any one of claims 1-124, wherein the composition does not comprise isolated mRNA.

126. The composition, formulation, method or use of any one of claims 1-125, wherein the LNP has an effective diameter of less than about 500 nanometers, optionally from about 5 to about 500 nanometers, optionally from about 10 to about 400 nanometers, optionally from about 20 to about 300 nanometers, or optionally from about 25 to about 250 nanometers.

127. The composition, formulation, method or use of claim 126, wherein the LNP has an effective diameter of less than about 250 nanometers.

128. The composition, formulation, method or use of claim 127, wherein the LNP has an effective diameter of less than about 125 nanometers.

129. The composition, formulation, method or use of claim 128, wherein the LNP has an effective diameter of from about 10 to about 110 nanometers

130. The composition, formulation, method or use of any one of claims 1-129, wherein the composition does not comprise a surfactant.
